(19) United States
(12) Patent Application Publication
(10) Pub. No.: US 2002/0035264 A1
(43) Pub. Date: Mar. 21, 2002

(54) Ophthalmic formulation of a selective cyclooxygenase-2 inhibitory drug

(76) Inventors: Tugrul T. Kararli, Skokie, IL (US); Rebanta Bandyopadhyay, Portage, MI (US); Satish K. Singh, Portage, MI (US); Leslie C. Hawley, Kalamazoo, MI (US)

Correspondence Address: FLYNN, THIEL, BOUTELL & TANIS, P.C., 2026 Rambling Road, Kalamazoo, MI, 49008-1699, US

(21) Appl. No.: 09/904,098

(22) Filed: Jul. 12, 2001

Related U.S. Application Data
(60) Provisional application No. 60/218,101, filed on Jul. 13, 2000.
(60) Provisional application No. 60/279,285, filed on Mar. 28, 2001.
(60) Provisional application No. 60/294,838, filed on May 31, 2001.
(60) Provisional application No. 60/296,388, filed on Jun. 6, 2001.

(51) Int. Cl. A61K 31/435; A61K 31/44; A61K 31/415

(52) U.S. Cl. 546/300; 546/301; 564/81; 548/377.1; 514/406; 514/351; 514/603

(57) ABSTRACT

There is provided a pharmaceutical composition suitable for topical administration to an eye which contains a selective COX-2 inhibitory drug or nanoparticles of a drug of low water solubility, in a concentration effective for treatment and/or prophylaxis of a disorder in the eye, and one or more ophthalmically acceptable excipients that reduce rate of removal from the eye such that the composition has an effective residence time of about 2 to about 24 hours. Also provided is a method of treating and/or preventing a disorder in an eye, the method comprising administering to the eye a composition of the invention.

This application claims the priority of U.S. Provisional Patent Application Ser. No. 60/218 101, filed Jul. 13, 2000, Ser. No. 60/279 285, filed on Mar. 28, 2001, Ser. No. 60/294 838, filed May 31, 2001 and Ser. No. 60/296 388, filed Jun. 6, 2001.

### FIELD OF THE INVENTION

The present invention relates to a pharmaceutical composition containing a drug of low water solubility and which is useful for topical application to an eye for treatment or prevention of an ophthalmic disease or disorder. In particular, the present invention relates to an ophthalmic formulation of a selective cyclooxygenase-2 inhibitory drug that can be applied to the eye for treatment or prevention of a cyclooxygenase-2 mediated ophthalmic disease or disorder. The field of the present invention also includes therapeutic or prophylactic use of such a composition, and use of such a composition in preparation of a medicament.

### BACKGROUND OF THE INVENTION

Numerous compounds have been reported having therapeutically and/or prophylactically useful selective cyclooxygenase-2 inhibitory effect, and have been disclosed as having utility in treatment or prevention of specific cyclooxygenase-2 mediated disorders or of such disorders in general. Among such compounds are a large number of substituted pyrazolyl benzenesulfonamides as reported in U.S. Pat. No. 5,466,823 to Talley et al., including for example 4-[5-(4-methylphenyl)-3-(trifluoromethyl)-1H-pyrazol-1-yl]benzenesulfonamide, referred to herein as “celecoxib”, and 4-[5-(3-fluoro-4-methoxyphenyl)-3-difluoromethyl)-1H-pyrazol-1-yl]benzenesulfonamide, referred to herein as “deracoxib”. Celecoxib has the structure shown in formula (I):

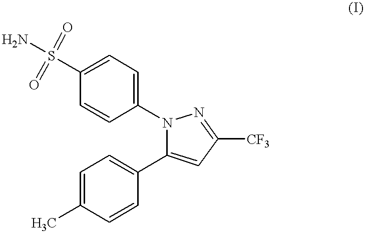

and deracoxib has the structure shown in formula (II):

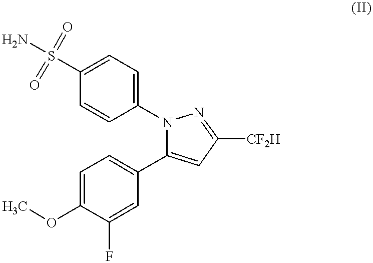

Other compounds reported to have therapeutically and/or prophylactically useful selective cyclooxygenase-2 inhibitory effect are substituted isoxazolyl benzenesulfonamides (see, e.g., U.S. Pat. No. 5,633,272 to Talley et al.) including, for example, 4-[5-methyl-3-phenylisoxazol-4-yl]-benzenesulfonamide, also referred to herein as “valdecoxib”, which has the structure shown in formula (III):

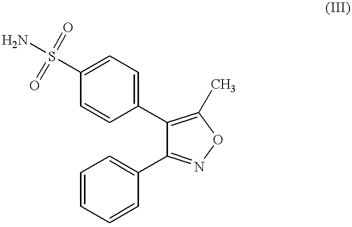

Parecoxib, is described in U.S. Pat. No. 5,932,598 to Talley et al. as one of a class of water-soluble prodrugs of selective COX-2 inhibitory drugs. Parecoxib, which has the structure shown in formula (IV) below, rapidly converts to the substantially water-insoluble selective COX-2 inhibitory drug valdecoxib following administration to a subject.

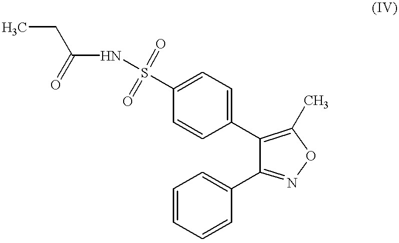

Still other compounds reported to have therapeutically and/or prophylactically useful selective cyclooxygenase-2 inhibitory effect are substituted (methylsulfonyl)-phenyl furanones (see, e.g., U.S. Pat. No. 5,474,995 to Ducharme et al.) including, for example, the compound 3-phenyl-4-[4-(methylsulfonyl)phenyl]-5H-furan-2-one, referred to herein as “rofecoxib”, which has the structure shown in formula (V):

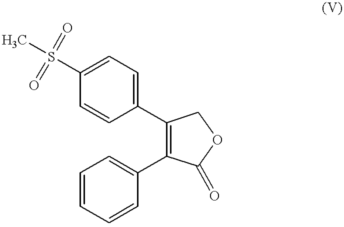

U.S. Pat. No. 5,981,576 to Belley et al. discloses additional(methylsulfonyl)phenyl furanones said to be useful as selective cyclooxygenase-2 inhibitory drugs, including 3-(1-cyclopropylmethoxy)-5,5-dimethyl-4-[4-(methylsulfonyl)phenyl]-5H-furan-2-one and 3-(1-cyclopropylethoxy)-5,5-dimethyl-4-[4-(methylsulfonyl)phenyl]-5H-furan-2-one.

U.S. Pat. No. 5,861,419 to Dube et al. discloses substituted pyridines said to be useful as selective cyclooxygenase-2 inhibitory drugs including, for example, 5-chloro-3-(4-methylsulfonyl)phenyl-2-(2-methyl-5-pyridinyl)pyridine, hereinafter referred to as “etoricoxib”, which has the structure shown in formula (VI )

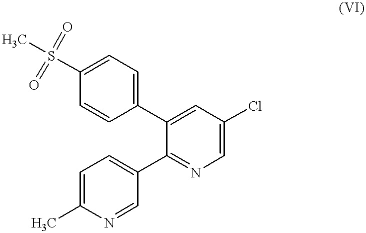

European Patent Application No. 0 863 134 discloses 2-(3,5-difluorophenyl)-3-[4-(methylsulfonyl)phenyl]-2-cyclopenten-1-one as a compound said to be useful as a selective cyclooxygenase-2 inhibitory drug.

U.S. Pat. No. 6,034,256 to Carter et al. discloses a series of benzopyrans said to be useful as selective COX-2 inhibitory drugs, including the compound (S)-6,8-dichloro-2-(trifluoromethyl)-2H-1-benzopyran-3-carboxylic acid (VII).

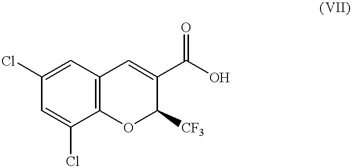

International Patent Publication No. WO 00/24719 filed by Abbott Laboratories discloses substituted pyridazinones said to be useful as selective COX-2 inhibitory drugs, including the compound 2-(3,4-difluorophenyl)-4-(3-hydroxy-3-methyl-1-butoxy)-5-[4-(methylsulfonyl)phenyl]-3-(2H)-pyridazinone.

Many patents relating to selective cyclooxygenase-2 inhibitory compounds, including those cited above, disclose utility of such compounds for relief of inflammation and inflammation-associated disorders. For example, above-cited U.S. Pat. Nos. 5,633,272 and 5,760,068 suggest that the subject compounds of these patents would be useful in treating inflammation in a long list of diseases, including conjunctivitis.

Masferrer & Kulkarni (1997), in Survey of Ophthalmology 41, Supplement 2, S35-S40, further teach utility of selective cyclooxygenase-2 inhibitory drugs in treating ocular inflammation, and suggest oral use of such drugs for this purpose.

International Patent Publication No. WO 00/32189 discloses orally deliverable compositions of celecoxib having utility in treatment of ophthalmic diseases such as retinitis, conjunctivitis, retinopathies, uveitis and ocular photophobia, and of acute injury to eye tissue. It is further disclosed therein that the subject orally deliverable compositions are useful for treatment of corneal graft rejection, ocular neovascularization, retinal neovascularization including that following injury or infection, diabetic retinopathy, macular degeneration, retrolental fibroplasia and neovascular glaucoma.

Formulations of nonsteroidal anti-inflammatories (hereinafter, “NSAIDs”) designed for topical application to the eye for relief of inflammation are known in the art. For example, a racemic mixture of R(+) and S(−) ketorolac tromethamine is formulated commercially as a sterile isotonic 0.5% aqueous solution in the product Acular® of Allergan, Inc., which is indicated for temporary relief of ocular itching due to seasonal allergic conjunctivitis, and for treatment of postoperative inflammation following cataract surgery. See Physicians' Desk Reference, 54th Edition (2000), pp. 491-492.

U.S. Pat. No. 4,474,751 to Haslam et al., incorporated herein by reference, discloses liquid aqueous ophthalmic compositions comprising a drug, preferably a water-soluble drug, together with 10% to 50% by weight of a thermosetting polymer that forms a gel at a human body temperature. Upon placement of such a liquid composition in an eye, a gel is formed thereby retarding loss of the drug from the eye by lacrimal drainage.

U.S. Pat. No. 4,861,760 to Mazuel & Friteyre, incorporated herein by reference, discloses a liquid in situ gelling composition said to be suitable for ophthalmic use. The composition contains in aqueous solution a polysaccharide that undergoes liquid-gel phase transition in response to ionic strength of tear fluid. A suitable polysaccharide is gellan gum, which can be used in a concentration of 0.1% to 2% by weight of the composition.

U.S. Pat. No. 5,587,175 to Viegas et al., incorporated herein by reference, discloses further liquid in situ gelling compositions said to be suitable for ophthalmic use. These compositions contain an ionic polysaccharide, for example gellan gum, alginate gum or chitosan, and a film-forming agent, for example hydroxypropyl methylcellulose, carboxymethylcellulose, sodium chondroitin sulfate, sodium hyaluronate, polyvinylpyrrolidone, etc. The compositions are pH buffered to match pH of tear fluid. Gelling is said to occur upon contact with calcium ions.

U.S. Pat. No. 6,174,524 to Bawa et al., incorporated herein by reference, discloses in situ gelling compositions, comprising xanthan gum for ophthalmic use. The gelling is said to be due, at least in part, to an interaction with the lysozyme component of tear fluid.

Each of the above cited U.S. Pat. No. 4,474,751, No. 4,861,760 and No. 5,587,175 suggests that the disclosed in situ gelling compositions can be used for ophthalmic delivery of anti-inflammatories such as indomethacin or sulindac.

U.S. Pat. No. 5,192,535 to Davis et al., incorporated herein by reference, discloses liquid compositions said to be suitable for use as eye drops, utilizing a different in situ gelling mechanism. These compositions contain a lightly cross-linked carboxyl-containing polymer such as polycarbophil and have a pH of about 3.0 to about 6.5. Upon placement of such a composition in an eye, contact with lacrimal fluid having a pH of about 7.2 to about 7.4 is said to result in gelling and consequent increase of residence time in the eye, permitting sustained release of a drug contained in the composition. Drugs for which such a composition is said to be useful include anti-inflammatories such as ibuprofen, flurbiprofen and naproxen and esters thereof; also ketorolac and suprofen.

U.S. Pat. No. 5,876,744 to Della Valle et al., incorporated herein by reference, discloses bioadhesive and mucoadhesive compositions, including some said to be useful as ophthalmic compositions, comprising mixtures of synthetic polymers such as polycarbophil and polyvinyl alcohol and biopolymers such as alginic acid, hyaluronic acid and dermatan sulfate. Such compositions are said to be capable of increasing contact time with a treated eye of specific drugs, for example anti-inflammatories.

U.S. Pat. No. 5,814,655 to Patel et al., incorporated herein by reference, discloses topical ophthalmic formulations of an NSAID such as diclofenac, suprofen or flurbiprofen wherein pH and concentration of the NSAID are such that a therapeutic amount of the NSAID is present in suspension and a therapeutic amount of the NSAID is present in solution.

Disclosure of topical ophthalmic formulations of an NSAID, for example diclofenac, ibuprofen, flurbiprofen, naproxen, ketorolac or suprofen, is also found individually in the references cited immediately below, which are incorporated herein by reference.

U.S. Pat. No. 4,559,343 to Han et al.

U.S. Pat. No. 4,960,799 to Nagy.

U.S. Pat. No. 4,829,083 to Doulakas.

U.S. Pat. No. 4,829,088 to Doulakas.

U.S. Pat. No. 5,110,493 to Cherng-Chyi et al.

International Patent Publication No. WO 95/03784.

International Patent Publication No. WO 95/18604.

International Patent Publication No. WO 95/31968.

Vulovic et al. (1989) Int. J. Pharmaceut. 55, 123-128.

Weisweiler et al. (1988) J. Clin. Res. Drug Development 2(4), 233-239.

International Patent Publication No. WO 99/59634, incorporated herein by reference, discloses eye drops containing a selective cyclooxygenase-2 inhibitory drug selected from etodolac, N-(2-(cyclohexyloxy)-4-nitrophenyl)-methanesulfonamide and meloxicam.

International Patent Publication No. WO 00/18387 filed by Alcon Laboratories, Inc, incorporated herein by reference, discloses ophthalmic compositions comprising an oxazolidinone antimicrobial agent and an anti-inflammatory agent. It is indicated that the anti-inflammatory agent can be any of a very broad list of agents, including selective cyclooxygenase-2 inhibitory drugs such as celecoxib. The anti-inflammatory agent is said to be present in such a composition in a concentration “sufficient to reduce inflammation” following topical application to the targeted tissues. The application describes examples of application of 1-4 drops of a solution or suspension, or a comparable amount of an ointment, gel or other solid or semisolid composition, 1-4 times a day, wherein the composition contains such an anti-inflammatory agent in a concentration of about 0.01% to about 1% by weight.

International Patent Publication No. WO 00/25771 for an application by Synphora AB, incorporated herein by reference, discloses ophthalmic compositions comprising a prostaglandin analog such as latanoprost and an anti-inflammatory agent. The anti-inflammatory agent is said to reduce iridial pigmentation during topical prostaglandin therapy for glaucoma. The anti-inflammatory agent can be any of a broad list of agents, including celecoxib and rofecoxib.

WO 00/18387 and WO 00/25771 cited above do not address the problems of formulating an ophthalmically acceptable composition comprising a selective cyclooxygenase-2 inhibitory drug. Such problems are particularly acute for selective cyclooxygenase-2 inhibitory drugs of low water solubility, including celecoxib, deracoxib, valdecoxib, rofecoxib, etoricoxib and 2-(3,5-difluorophenyl)-3-[4-(methylsulfonyl)phenyl]-2-cyclopenten-1-one. Furthermore, the composition must generally be formulated in a way that provides continuous delivery of the drug to an eye in order to be therapeutically effective against a cyclooxygenase-2 mediated disorder of the eye.

A need therefore remains for a method of topically treating and/or preventing cyclooxygenase-2 mediated disorders of the eye. A special need exists for such a method having its therapeutic and/or prophylactic effect through selective inhibition of cyclooxygenase-2 (COX-2), without the undesirable side-effects associated with inhibition of cyclooxygenase-1 (COX-1) that can occur with conventional NSAIDs. A particular need exists for an ophthalmically acceptable formulation of a selective COX-2 inhibitory drug, particularly one of low water solubility, that is therapeutically and/or prophylactically effective when administered topically to the eye. A further particular need exists for such a formulation that delivers the drug continuously to the eye over a prolonged period of time, for example over at least about 2 hours, preferably longer.

These and other needs will be seen to be met by the invention now described.

### SUMMARY OF THE INVENTION

The present invention provides a pharmaceutical composition suitable for topical administration to an eye, the composition comprising nanoparticles of a drug of low water solubility in a concentration effective for treatment and/or prophylaxis of a disorder in the eye, and at least one ophthalmically acceptable excipient ingredient that preferably reduce the rate of removal of the composition from the eye by lacrimation such that the composition has an effective residence time in an eye of about 2 to about 24 hours, after being applied thereto.

The present invention also provides a pharmaceutical composition suitable for topical administration to an eye, the composition comprising a selective COX-2 inhibitory drug in a concentration effective for treatment and/or prophylaxis of a COX-2 mediated disorder in the eye, and one or more ophthalmically acceptable excipient ingredients that preferably reduce rate of removal of the composition from the eye by lacrimation such that the composition has an effective residence time in the eye of about 2 to about 24 hours.

As used herein, the term “lacrimation” refers to the production of tear fluid. Matter is typically removed from eyes by external wash-out, by lacrimal drainage into the nasopharyngeal cavity via the nasolacrimal ducts, or by a combination of the two means. By “effective residence time” herein is meant a period of time following application of the composition to the eye during which a substantial portion of the applied composition remains in situ even in the presence of lacrimation and/or external wash-out, and during which the drug is released therefrom in a therapeutically or prophylactically effective amount to tissues of the eye and/or to fluids secreted thereby.

The composition, therefore provides sustained release at an effective concentration over a period of at least about 2 hours. Optionally, a portion of the selective COX-2 inhibitory drug can be present in the composition in immediate-release form so that the composition provides a combination of immediate and sustained release (herein referred to as “dual release”) of the drug.

The invention also provides a method of preparing a medicament for treating or preventing a COX-2 mediated disorder in an eye, using a composition as described above.

The present invention provides a method of treating or preventing a disorder in an eye, the method comprising application of a composition as described above in a therapeutically or prophylactically effective dose.

Also embraced by the present invention is a method of treating or preventing a COX-2 mediated disorder in an eye, the method comprising application to the eye of a composition as described above in a therapeutically or prophylactically effective dose.

COX-2 mediated disorders of the eye for which the method is useful include without limitation inflammatory disorders such as endophthalmitis, episcleritis, retinitis, iriditis, cyclitis, choroiditis, keratitis, conjunctivitis and blepharitis, including inflammation of more than one part of the eye, e.g., retinochoroiditis, iridocyclitis, iridocyclochoroiditis (also known as uveitis), keratoconjunctivitis, blepharoconjunctivitis, etc.; other COX-2 mediated retinopathies including diabetic retinopathy; ocular tumors; ocular photophobia; acute trauma of any tissue of the eye including postsurgical trauma, e.g., following cataract or corneal transplant surgery; postsurgical ocular inflammation; intraoperative miosis; corneal graft rejection; ocular, for example retinal, neovascularization including that following injury or infection; macular degeneration; cystoid macular edema; retrolental fibroplasia; neovascular glaucoma; ocular pain; and COX-2 mediated side effects from ocular prostaglandin therapy including increased iridial pigmentation, disruption of the blood aqueous barrier and cystoid macular edema.

It is understood that certain COX-2 mediated disorders of the eye are disorders of surface tissues such as the conjunctiva, and that topical application of a selective COX-2 inhibitory drug to the eye, therefore, results in delivery of the drug directly to its site of action in the case of such disorders. Other COX-2 mediated disorders of the eye are disorders of internal tissues such as the retina, in which case the drug has to move from the locus of administration to the targeted tissue. Administration of a composition of the invention to the eye generally results in direct contact of the drug with the cornea, through which at least a portion of the administered drug passes. The term “topical” as applied herein to ocular administration of a composition of the invention will be understood to embrace administration followed by corneal absorption as well as administration directly to a targeted surface tissue of the eye such as open tissues in connection with eye surgery or postsurgical treatment.

What constitutes a “concentration effective for treatment and/or prophylaxis of a COX-2 mediated disorder” depends, among other factors, on the particular drug being administered; the residence time provided by the particular formulation of the drug; the species, age and body weight of the subject; the particular ophthalmic condition for which treatment or prophylaxis is sought; and the severity of the condition. In the case of celecoxib, an effective concentration in a composition of the invention for topical administration to an eye will generally be found in the range from about 0.1% to about 50% weight/volume. For selective COX-2 inhibitory drugs other than celecoxib, an appropriate concentration range is one that is therapeutically equivalent to the celecoxib concentration range indicated above.

A composition of the invention is preferably formulated as an in situ gellable aqueous liquid, and can be administered as eye drops. Typically each drop of the composition, generated by a conventional dispensing means, has a volume of about 15 to about 40 μl. From 1 to about 6 such drops typically provides a suitable dose of the selective COX-2 inhibitory drug. Where the composition is administered in a form other than eye drops, for example as an ophthalmic ointment or as a solid implant, an equivalent dose is provided. Such a dose can be administered as needed, but typically administration to the eye 1 to about 4 times per day, in most cases 1 or 2 times a day, provides adequate continuing relief or prevention of the ophthalmic disorder indicated.

The term “ophthalmically acceptable” with respect to a formulation, composition or ingredient herein means having no persistent detrimental effect on the treated eye or the functioning thereof, or on the general health of the subject being treated. It will be recognized that transient effects such as minor irritation or a “stinging” sensation are common with topical ophthalmic administration of drugs and the existence of such transient effects is not inconsistent with the formulation, composition or ingredient in question being “ophthalmically acceptable” as herein defined. However, preferred formulations, compositions and ingredients are those that cause no substantial detrimental effect, even of a transient nature.

By contrast with therapeutic and prophylactic methods involving NSAIDs lacking selectivity for inhibition of COX-2, highly effective relief or prevention of COX-2 mediated ophthalmic disorders can be obtained with greatly reduced risk of the side-effects commonly associated with COX-1 inhibition. Thus the method of the present invention is particularly suitable where conventional NSAIDs are contraindicated, for example in patients with peptic ulcers, gastritis, regional enteritis, ulcerative colitis or diverticulitis, patients with a recurrent history of gastrointestinal lesions, patients with gastrointestinal bleeding, coagulation disorders including anemia such as hypothrombinemia, hemophilia and other bleeding problems, or kidney disease, patients prior to surgery, or patients taking anticoagulants.

A particular advantage over conventional NSAIDs for topical application to eyes is the lack of effect on baseline COX-1 mediated physiological functions including wound healing following eye surgery, and intraocular pressure control.

Further, previously disclosed ophthalmic compositions containing a selective COX-2 inhibitory drug have not been specified to be resistant to removal from a treated eye by lacrimation, and in particular an effective residence time of at least about 2 hours has not been specified. Yet, according to the present invention, such a residence time is believed to be critical in the case of at least the great majority of selective COX-2 inhibitory drugs. Without being bound by theory, it is believed that the criticality of a sufficiently long residence time arises at least in part from the following factors.

A first factor is the extremely low solubility in water of most selective COX-2 inhibitory drugs, including for example celecoxib, deracoxib, valdecoxib, rofecoxib, etoricoxib, 2-(3,5-difluorophenyl)-3-[4-(methylsulfonyl)phenyl]-2-cyclopenten-1-one and 2-(3,4-difluorophenyl)-4-(3-hydroxy-3-methyl-1-butoxy)-5-[4-(methylsulfonyl)phenyl]-3-(2H)-pyridazinone. In aqueous compositions, such drugs are typically present as dispersed particles, from which release is not instantaneous. Without the residence time provided by the present invention, an insufficient amount of the drug would be released before loss of the composition from the treated eye by lacrimal drainage.

A second factor is the need for sustained COX-2 inhibitory action. When a selective COX-2 inhibitory drug is administered orally, it is absorbed in the alimentary tract into the bloodstream and becomes systemically distributed throughout the body. Typically such a drug has a relatively long half-life in the bloodstream and repeat administration is generally not necessary for about 12 to about 24 hours or more. By contrast, in a topical administration method as provided by the present invention, the dosage is generally insufficient to lead to a therapeutically or prophylactically effective blood serum concentration and sustained effectiveness is therefore dependent on a depot of the drug remaining in situ at the locus of application.

The very low dose permitted by the composition and method of the invention, by comparison with normal orally administered doses of selective COX-2 inhibitory drugs, is a further advantage of the invention.

Formulations of the invention are contemplated to be useful for any drug, of low water solubility, for which ophthalmic administration is desired.

Other features and advantages of the invention will be in part apparent and in part pointed out hereinafter.

### BRIEF DESCRIPTION OF THE DRAWINGS

FIG. 1 is a graph illustrating the pharmacokinetic results of ocular versus oral delivery for the concentration of valdecoxib in the conjunctiva plotted against time;

FIG. 2 is a graph illustrating the valdecoxib concentration in the conjunctiva, cornea, aqueous humor and plasma four hours after oral administration of the drug; and

FIG. 3 is a graph illustrating the valdecoxib concentration in the conjunctiva, cornea, aqueous humor and plasma four hours after ocular administration of the drug.

### DETAILED DESCRIPTION OF THE INVENTION

As indicated above, the invention provides a pharmaceutical composition suitable for topical administration to an eye. The composition comprises a selective COX-2 inhibitory drug or a salt or prodrug thereof in a concentration effective for treatment and/or prophylaxis of a COX-2 mediated disorder in the eye, and one or more ophthalmically acceptable excipient ingredients that reduce rate of removal of the composition from the eye by lacrimation, such reduction in rate of removal including rendering the composition resistant to removal from the eye by lacrimation. By virtue at least in part of this reduced rate of removal by lacrimation, the composition has an effective residence time in the eye of about 2 to about 24 hours.

In one embodiment, the selective COX-2 inhibitory drug is of low water solubility. Low water solubility herein is defined as water solubility of not more than about 10 mg/ml, preferably not more than about 5 mg/ml, for example not more than about 1 mg/ml.

The selective COX-2 inhibitory drug can be any such drug known in the art, including, without limitation, compounds disclosed in the patents and publications listed below, each of which is individually incorporated herein by reference.

U.S. Pat. No. 5,344,991 to Reitz & Li.

U.S. Pat. No. 5,380,738 to Norman et al.

U.S. Pat. No. 5,393,790 to Reitz et al.

U.S. Pat. No. 5,401,765 to Lee.

U.S. Pat. No. 5,418,254 to Huang & Reitz.

U.S. Pat. No. 5,420,343 to Koszyk & Weier.

U.S. Pat. No. 5,434,178 to Talley & Rogier.

U.S. Pat. No. 5,436,265 to Black et al.

Above-cited U.S. Pat. No. 5,466,823.

Above-cited U.S. Pat. No. 5,474,995.

U.S. Pat. No. 5,475,018 to Lee & Bertenshaw.

U.S. Pat. No. 5,486,534 to Lee et al.

U.S. Pat. No. 5,510,368 to Lau et al.

U.S. Pat. No. 5,521,213 to Prasit et al.

U.S. Pat. No. 5,536,752 to Ducharme et al.

U.S. Pat. No. 5,543,297 to Cromlish et al.

U.S. Pat. No. 5,547,975 to Talley et al.

U.S. Pat. No. 5,550,142 to Ducharme et al.

U.S. Pat. No. 5,552,422 to Gauthier et al.

U.S. Pat. No. 5,585,504 to Desmond et al.

U.S. Pat. No. 5,593,992 to Adams et al.

U.S. Pat. No. 5,596,008 to Lee.

U.S. Pat. No. 5,604,253 to Lau et al.

U.S. Pat. No. 5,604,260 to Guay & Li.

U.S. Pat. No. 5,616,458 to Lipsky et al.

U.S. Pat. No. 5,616,601 to Khanna et al.

U.S. Pat. No. 5,620,999 to Weier et al.

Above-cited U.S. Pat. No. 5,633,272.

U.S. Pat. No. 5,639,780 to Lau et al.

U.S. Pat. No. 5,643,933 to Talley et al.

U.S. Pat. No. 5,658,903 to Adams et al.

U.S. Pat. No. 5,668,161 to Talley et al.

U.S. Pat. No. 5,670,510 to Huang & Reitz.

U.S. Pat. No. 5,677,318 to Lau.

U.S. Pat. No. 5,681,842 to Dellaria & Gane.

U.S. Pat. No. 5,686,460 to Nicolai et al.

U.S. Pat. No. 5,686,470 to Weier et al.

U.S. Pat. No. 5,696,143 to Talley et al.

U.S. Pat. No. 5,710,140 to Ducharme et al.

U.S. Pat. No. 5,716,955 to Adams et al.

U.S. Pat. No. 5,723,485 to Gungor & Teulon.

U.S. Pat. No. 5,739,166 to Reitz et al.

U.S. Pat. No. 5,741,798 to Lazer et al.

U.S. Pat. No. 5,756,499 to Adams et al.

U.S. Pat. No. 5,756,529 to Isakson & Talley.

U.S. Pat. No. 5,776,967 to Kreft et al.

U.S. Pat. No. 5,783,597 to Beers & Wachter.

U.S. Pat. No. 5,789,413 to Black et al.

U.S. Pat. No. 5,807,873 to Nicolai & Teulon.

U.S. Pat. No. 5,817,700 to Dube et al.

U.S. Pat. No. 5,8301911 to Failli et al.

U.S. Pat. No. 5,849,943 to Atkinson & Wang.

U.S. Pat. No. 5,859,036 to Sartori et al.

Above-cited U.S. Pat. No. 5,861,419.

U.S. Pat. No. 5,866,596 to Sartori & Teulon.

U.S. Pat. No. 5,869,524 to Failli.

U.S. Pat. No. 5,869,660 to Adams et al.

U.S. Pat. No. 5,883,267 to Rossen et al.

U.S. Pat. No. 5,892,053 to Zhi et al.

U.S. Pat. No. 5,922,742 to Black et al.

U.S. Pat. No. 5,929,076 to Adams & Garigipati.

Above-cited U.S. Pat. No. 5,932,598.

U.S. Pat. No. 5,935,990 to Khanna et al.

U.S. Pat. No. 5,945,539 to Haruta et al.

U.S. Pat. No. 5,958,978 to Yamazaki et al.

U.S. Pat. No. 5,968,958 to Guay et al.

U.S. Pat. No. 5,972,950 to Nicolai & Teulon.

U.S. Pat. No. 5,973,191 to Marnett & Kalgutkar.

Above-cited U.S. Pat. No. 5,981,576.

U.S. Pat. No. 5,994,381 to Haruta et al.

U.S. Pat. No. 6,002,014 to Haruta et al.

U.S. Pat. No. 6,004,960 to Li et al.

U.S. Pat. No. 6,005,000 to Hopper et al.

U.S. Pat. No. 6,020,343 to Belley et al.

U.S. Pat. No. 6,020,347 to DeLaszlo & Hagmann.

Above-cited U.S. Pat. No. 6,034,256.

U.S. Pat. No. 6,040,319 to Corley et al.

U.S. Pat. No. 6,040,450 to Davies et al.

U.S. Pat. No. 6,046,208 to Adams et al.

U.S. Pat. No. 6,046,217 to Friesen et al.

U.S. Pat. No. 6,057,319 to Black et al.

U.S. Pat. No. 6,063,804 to De Nanteuil et al.

U.S. Pat. No. 6,063,807 to Chabrier de Lassauniere & Broquet.

U.S. Pat. No. 6,071,954 to LeBlanc et al.

U.S. Pat. No. 6,077,868 to Cook et al.

U.S. Pat. No. 6,077,869 to Sui & Wachter.

U.S. Pat. No. 6,083,969 to Ferro et al.

U.S. Pat. No. 6,096,753 to Spohr et al.

U.S. Pat. No. 6,133,292 to Wang et al.

International Patent Publication No. WO 94/15932.

International Patent Publication No. WO 96/19469.

International Patent Publication No. WO 96/26921.

International Patent Publication No. WO 96/31509.

International Patent Publication No. WO 96/36623.

International Patent Publication No. WO 96/38418.

International Patent Publication No. WO 97/03953.

International Patent Publication No. WO 97/10840.

International Patent Publication No. WO 97/13755.

International Patent Publication No. WO 97/13767.

International Patent Publication No. WO 97/25048.

International Patent Publication No. WO 97/30030.

International Patent Publication No. WO 97/34882.

International Patent Publication No. WO 97/46524.

International Patent Publication No. WO 98/04527.

International Patent Publication No. WO 98/06708.

International Patent Publication No. WO 98/07425.

International Patent Publication No. WO 98/17292.

International Patent Publication No. WO 98/21195.

International Patent Publication No. WO 98/22457.

International Patent Publication No. WO 98/32732.

International Patent Publication No. WO 98/41516.

International Patent Publication No. WO 98/43966.

International Patent Publication No. WO 98/45294.

International Patent Publication No. WO 98/47871.

International Patent Publication No. WO 99/01130.

International Patent Publication No. WO 99/01131.

International Patent Publication No. WO 99/01452.

International Patent Publication No. WO 99/01455.

International Patent Publication No. WO 99/10331.

International Patent Publication No. WO 99/10332.

International Patent Publication No. WO 99/11605.

International Patent Publication No. WO 99/12930.

International Patent Publication No. WO 99/14195.

International Patent Publication No. WO 99/14205.

International Patent Publication No. WO 99/15505.

International Patent Publication No. WO 99/23087.

International Patent Publication No. WO 99/24404.

International Patent Publication No. WO 99/25695.

International Patent Publication No. WO 99/35130.

International Patent Publication No. WO 99/61016.

International Patent Publication No. WO 99/61436.

International Patent Publication No. WO 99/62884.

International Patent Publication No. WO 99/64415.

International Patent Publication No. WO 00/01380.

International Patent Publication No. WO 00/08024.

International Patent Publication No. WO 00/10993.

International Patent Publication No. WO 00/13684.

International Patent Publication No. WO 00/18741.

International Patent Publication No. WO 00/18753.

International Patent Publication No. WO 00/23426.

International Patent Publication No. WO 00/24719.

International Patent Publication No. WO 00/26216.

International Patent Publication No. WO 00/31072.

International Patent Publication No. WO 00/40087.

International Patent Publication No. WO 00/56348.

Europea n Patent Application No. 0 799 823.

European Patent Application No. 0 846 689.

Above-cited European Patent Application No. 0 863 134.

European Patent Application No. 0 985 666.

Compositions of the invention are especially useful for compounds having the formula (VII):

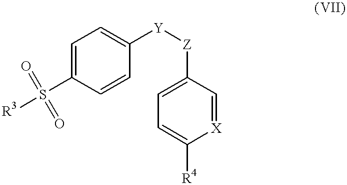

where R³ is a methyl or amino group, R⁴ is hydrogen or a C₁₋₄ alkyl or alkoxy group, X is N or CR⁵ where R⁵ is hydrogen or halogen, and Y and Z are independently carbon or nitrogen atoms defining adjacent atoms of a five- to six-membered ring that is unsubstituted or substituted at one or more positions with oxo, halo, methyl or halomethyl groups. Preferred such five- to six-membered rings are cyclopentenone, furanone, methylpyrazole, isoxazole and pyridine rings substituted at no more than one position. Also useful are prodrugs that provide such selective COX-2 inhibitory compounds upon administration, for example parecoxib, which is a prodrug of valdecoxib.

Illustratively, celecoxib, deracoxib, valdecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid and 2-(3,4-difluorophenyl)-4-(3-hydroxy-3-methyl-1-butoxy)-5-[4-(methylsulfonyl)phenyl]-3-(2H)-pyridazinone, more particularly celecoxib, valdecoxib, rofecoxib and etoricoxib, still more particularly valdecoxib and etoricoxib, and most particularly valdecoxib, are useful in the method and composition of the invention.

The invention is illustrated herein with particular reference to celecoxib, and it will be understood that any other selective COX-2 inhibitory compound can, if desired, be substituted in whole or in part for celecoxib, with appropriate adjustment in concentration and dosage ranges, in the compositions and methods herein described.

Selective COX-2 inhibitory drugs used in the method and composition of the invention can be prepared by a process known per se, in the case of celecoxib, for example, by processes described in U.S. Pat. No. 5,466,863 to Talley et al. or in U.S. Pat. No. 5,892,053 to Zhi & Newaz, both incorporated herein by reference. Other selective COX-2 inhibitory drugs can be prepared by processes known per se, including processes set forth in patent publications disclosing such drugs; for example in the case of valdecoxib in above-cited U.S. Pat. No. 5,633,272, and in the case of rofecoxib in above-cited U.S. Pat. No. 5,474,995.

Preferably the composition has an effective residence time in the eye of about 3 to about 24 hours, more preferably about 4 to about 24 hours and most preferably about 6 to about 24 hours.

A composition of the invention can illustratively take the form of a liquid wherein the drug is present in solution, in suspension or both. The term “solution/suspension” herein refers to a liquid composition wherein a first portion of the drug is present in solution and a second portion of the drug is present in particulate form, in suspension in a liquid matrix. A liquid composition herein includes a gel. Preferably the liquid composition is aqueous. Alternatively, the composition can take the form of an ointment. The composition of the present invention can also be delivered by electrophoresis, electroporation or iontophoresis.

As a further alternative, the composition can take the form of a solid article that can be inserted between the eye and eyelid or in the conjunctival sac, where it releases the drug as described, for example, in U.S. Pat. No. 3,863,633 and U.S. Pat. No. 3,868,445, both to Ryde & Ekstedt, incorporated herein by reference. Release is to the lacrimal fluid that bathes the surface of the cornea, or directly to the cornea itself, with which the solid article is generally in intimate contact. Solid articles suitable for implantation in the eye in such fashion are generally composed primarily of polymers and can be biodegradable or non-biodegradable. Biodegradable polymers that can be used in preparation of ocular implants carrying a selective COX-2 inhibitory drug in accordance with the present invention include without restriction aliphatic polyesters such as polymers and copolymers of poly(glycolide), poly(lactide), poly(c-caprolactone), poly(hydroxybutyrate) and poly(hydroxyvalerate), polyamino acids, polyorthoesters, polyanhydrides, aliphatic polycarbonates and polyether lactones. Illustrative of suitable non-biodegradable polymers are silicone elastomers.

In a presently preferred embodiment, the composition is an aqueous solution, suspension or solution/suspension, which can be presented in the form of eye drops. By means of a suitable dispenser, a desired dosage of the drug can be metered by administration of a known number of drops into the eye. For example, for a drop volume of 25 μl, administration of 1-6 drops will deliver 25-150 μl of the composition. Aqueous compositions of the invention preferably contain from about 0.01% to about 50%, more preferably about 0.1% to about 20%, still more preferably about 0.2% to about 10%, and most preferably about 0.5% to about 5%, weight/volume of the selective COX-2 inhibitory drug. In one embodiment, a composition of the invention contains a concentration of the selective COX-2 inhibitory drug that is therapeutically or prophylactically equivalent to a celecoxib weight/volume concentration of about 0.1% to about 50%, preferably about 0.5% to about 20%, and most preferably about 1% to about 10%. In another embodiment, a composition of the invention has relatively high loading of the drug and is suitable for a relatively long residence time in a treated eye. In this embodiment the weight/volume concentration of the drug in the composition is about 1.3% to about 50%, preferably about 1.5% to about 30%, and most preferably about 2% to about 20%, for example about 2% to about 10%.

Preferably no more than 3 drops, more preferably no more than 2 drops, and most preferably no more than 1 drop, each of about 15 to about 40 μl, preferably about 20 to about 30 μl, for example about 25 μl, should contain the desired dose of the drug for administration to an eye. Administration of a larger volume to the eye risks loss of a significant portion of the applied composition by lacrimation.

Aqueous compositions of the invention have ophthalmically compatible pH and osmolality.

In an aqueous suspension or solution/suspension composition of a preferred embodiment of the invention, the selective COX-2 inhibitory drug is present predominantly in the form of nanoparticles, i.e., solid particles smaller than about 1 μm in their longest dimension. A benefit of this embodiment is more rapid release of the drug, and therefore more complete release during the residence time of the composition in a treated eye, than occurs with larger particle size. Another benefit is reduced potential for eye irritation by comparison with larger particle size. Reduced eye irritation in turn leads to a reduced tendency for loss of the composition from the treated eye by lacrimation, which is stimulated by such irritation.

In a related embodiment the drug preferably has a D₉₀ particle size of about 0.01 to about 200 μm, wherein about 25% to 100% by weight of the particles are nanoparticles. “D₉₀” is defined as a linear measure of diameter having a value such that 90% by volume of particles in the composition, in the longest dimension of the particles, are smaller than that diameter. For practical purposes a determination of D₉₀ based on 90% by weight rather than by volume is generally suitable.

In one embodiment substantially all of the drug particles in the composition are smaller than 1 μm, i.e., the percentage by weight of nanoparticles is 100% or close to 100%. The selective cyclooxygenase-2 inhibitory drug can be in crystalline or amorphous form in the nanoparticles. Processes for preparing nanoparticles that involve milling or grinding typically provide the drug in crystalline form, whereas processes that involve precipitation from solution typically provide the drug in amorphous form.

Nanoparticles comprising or consisting essentially of a selective COX-2 inhibitory drug of low water solubility can be prepared according to any process previously applied to preparation of other poorly water soluble drugs in form of nanoparticles. Suitable processes, without restriction, are illustratively disclosed for such other drugs in patents and publications listed below and incorporated herein by reference.

U.S. Pat. No. 4,826,689 to Violanto & Fischer.

Above-cited U.S. Pat. No. 5,145,684.

U.S. Pat. No. 5,298,262 to Na & Rajagopalan.

U.S. Pat. No. 5,302,401 to Liversidge et al.

U.S. Pat. No. 5,336,507 to Na & Rajagopalan.

U.S. Pat. No. 5,340,564 to Illig & Sarpotdar.

U.S. Pat. No. 5,346,702 to Na & Rajagopalan.

U.S. Pat. No. 5,352,459 to Hollister et al.

U.S. Pat. No. 5,354,560 to Lovrecich.

Above-cited U.S. Pat. No. 5,384,124.

U.S. Pat. No. 5,429,824 to June.

U.S. Pat. No. 5,503,723 to Ruddy et al.

U.S. Pat. No. 5,510,118 to Bosch et al.

U.S. Pat. No. 5,518,187 to Bruno et al.

U.S. Pat. No. 5,518,738 to Eickhoff et al.

U.S. Pat. No. 5,534,270 to De Castro.

U.S. Pat. No. 5,536,508 to Canal et al.

U.S. Pat. No. 5,552,160 to Liversidge et al.

U.S. Pat. No. 5,560,931 to Eickhoff et al.

U.S. Pat. No. 5,560,932 to Bagchi et al.

U.S. Pat. No. 5,565,188 to Wong et al.

U.S. Pat. No. 5,569,448 to Wong et al.

U.S. Pat. No. 5,571,536 to Eickhoff et al.

U.S. Pat. No. 5,573,783 to Desieno & Stetsko.

U.S. Pat. No. 5,580,579 to Ruddy et al.

U.S. Pat. No. 5,585,108 to Ruddy et al.

U.S. Pat. No. 5,587,143 to Wong.

U.S. Pat. No. 5,591,456 to Franson et al.

U.S. Pat. No. 5,622,938 to Wong.

U.S. Pat. No. 5,662,883 to Bagchi et al.

U.S. Pat. No. 5,665,331 to Bagchi et al.

U.S. Pat. No. 5,718,919 to Ruddy et al.

U.S. Pat. No. 5,747,001 to Wiedmann et al.

Above-cited International Patent Publication No. WO 93/25190.

International Patent Publication No. WO 96/24336.

International Patent Publication No. WO 97/14407.

International Patent Publication No. WO 98/35666.

International Patent Publication No. WO 99/65469.

International Patent Publication No. WO 00/18374.

International Patent Publication No. WO 00/27369.

International Patent Publication No. WO 00/30615.

One of ordinary skill in the art will readily adapt the processes therein described to preparation of a poorly water soluble selective COX-2 inhibitory drug in form of nanoparticles.

In one embodiment of the invention, nanoparticles of a selective COX-2 inhibitory drug are prepared by a milling process, preferably a wet milling process in presence of a surface modifying agent that inhibits aggregation and/or crystal growth of nanoparticles once created. In another embodiment of the invention, nanoparticles of a selective COX-2 inhibitory drug are prepared by a precipitation process, preferably a process of precipitation in an aqueous medium from a solution of the drug in a non-aqueous solvent. The non-aqueous solvent can be a liquefied, e.g., supercritical, gas under pressure. Illustrative examples of these and other processes for preparing nanoparticles of a selective COX-2 inhibitory drug are presented with greater particularity below.

In one particular embodiment of the invention, nanoparticles are prepared by a process comprising the steps of (a) dispersing a selective COX-2 inhibitory drug and a surface modifying agent in a liquid dispersion medium; and (b) wet milling the resulting drug dispersion in presence of grinding media to result in crystalline nanoparticles of the drug having the surface modifying agent adsorbed on the surface thereof in an amount sufficient to maintain a weight average particle size of less than about 400 nm, substantially as disclosed in above-cited U.S. Pat. No. 5,145,684. The surface modifying agent inhibits aggregation of the nanoparticles and can be any of various polymers, low molecular weight oligomers, natural products, surfactants, etc. The nanoparticles in this and related embodiments are referred to herein as being composed of a nanocrystalline drug/surface modifier complex.

In another embodiment of the invention, if there are relatively high amounts of an amorphous phase in the drug composition, water-soluble polymeric excipients, such as povidone and modified celluloses, may be present in order to help prevent the occurrence of nucleation/crystallization.

In a related embodiment of the invention, a nanocrystalline drug/surface modifier complex prepared as described above comprises a purified surface modifying agent, for example a purified polymeric surfactant, to prevent particle aggregation during a subsequent sterilization step, substantially as disclosed in above-cited U.S. Pat. No. 5,352,459.

In another related embodiment of the invention, a nanocrystalline drug/surface modifier complex prepared as described above comprises as a surface modifying agent the surfactant p-isononylphenoxypoly(glycidol), substantially as disclosed in above-cited U.S. Pat. No. 5,340,564.

In another related embodiment of the invention, a nanocrystalline drug/surface modifier complex prepared as described above is associated with an anionic or cationic cloud point modifier to increase the cloud point of the surface modifying agent, substantially as described in above-cited U.S. Pat. No. 5,298,262 (cationic or anionic surfactant as cloud point modifier), U.S. Pat. No. 5,336,507 (charged phospholipid as cloud point modifier), or U.S. Pat. No. 5,346,702 (non-ionic cloud point modifier).

In another related embodiment of the invention, a nanocrystalline drug/surface modifier complex prepared as described above further comprises a cryoprotectant, for example a carbohydrate or sugar alcohol, in an amount sufficient to permit the nanoparticles to be lyophilized, substantially as described in above-cited U.S. Pat. No. 5,302,401. A preferred cryoprotectant of this embodiment is sucrose. The method of making nanoparticles having a surface modifier adsorbed on the surface thereof and a cryoprotectant associated therewith comprises contacting the nanoparticles with the cryoprotectant for a time and under conditions sufficient to permit lyophilization of the nanoparticles.

In another related embodiment of the invention, drug nanoparticles having a surface modifying agent adsorbed on the surface thereof in an amount sufficient to maintain a weight average particle size of less than about 400 nm are prepared by a process comprising the steps of (a) dispersing the drug in a liquid dispersion medium wherein the drug is insoluble; and (b) grinding the medium (e.g., in a dispersion mill) in the presence of rigid grinding media, wherein pH of the medium is maintained within a range of about 2 to about 6, substantially as disclosed in above-cited U.S. Pat. No. 5,552,160.

In another related embodiment of the invention, nanoparticles are prepared by a process comprising the steps of (a) providing a selective COX-2 inhibitory drug substance; (b) depyrogenating rigid grinding media, for example in an oven at about 200° C. to about 300° C. for about 6 to about 20 hours; mixing the drug substance and grinding media together and autoclaving at about 100° C. to about 150° C. for about 10 to about 60 minutes); and (c) adding a surface modifying agent (e.g., selected from polymers, low molecular weight oligomers, natural products and surfactants) to the resulting autoclaved drug substance followed by wet grinding to provide and maintain a weight average particle size of less than about 400 nm, substantially as disclosed in above-cited U.S. Pat. No. 5,534,270.

In another related embodiment of the invention, nanoparticles are prepared by a process comprising contacting a selective COX-2 inhibitory drug with a surface modifying agent (e.g., by adding the drug to a liquid medium comprising the surface modifying agent and wet grinding in a dispersion mill) for a time and under conditions sufficient to provide and maintain a weight average particle size of less than about 400 nm, substantially as described in above-cited U.S. Pat. No. 5,429,824. In this embodiment the surface modifying agent is a nonionic liquid polymer of the alkylaryl polyether alcohol type, for example tyloxapol. Optionally an additional surface modifying agent can be present.

In another related embodiment of the invention, nanoparticles are prepared by a process comprising the steps of (a) forming a premix of a selective COX-2 inhibitory drug and a surface modifier (e.g., selected from polymers, low molecular weight oligomers, surfactants, etc.) in a liquid dispersion medium (e.g., water, salt solution, ethanol, etc.); (b) transferring the premix to a microfluidizer having an interaction chamber capable of producing shear, impact, cavitation and attrition forces; (c) subjecting the premix to these forces at a temperature not exceeding about 40° C. and a fluid pressure of about 20,000 to about 200,000 kPa by passing the premix through the interaction chamber to reduce the particle size of the drug and to obtain a homogeneous slurry thereof; (d) collecting the slurry from the interaction chamber into a receiving tank; (e) reintroducing the slurry into the interaction chamber to further reduce particle size; and (f) repeating the collection and reintroduction steps until the weight average particle size of the drug is less than about 400 nm, substantially as disclosed in above-cited U.S. Pat. No. 5,510,118.

In another related embodiment of the invention, nanoparticles are prepared by a process comprising the steps of (a) milling (e.g., in a dispersion mill), optionally in the presence of an oil, a selective COX-2 inhibitory drug in the presence of surface modifying agents (e.g., gelatin, casein, lecithin, polyvinylpyrrolidone, tyloxapol, poloxamers, other block polymers, etc.) substantially as disclosed in above-cited U.S. Pat. No. 5,560,931. In this embodiment, the drug particles have a non-crosslinked modifier adsorbed on the surface thereof, and are suspended in an aqueous phase which is emulsified in a continuous oil phase. Weight average particle size is less than about 1000 nm. The oil phase can be oleic acid, as disclosed in above-cited U.S. Pat. No. 5,571,536.

In another related embodiment of the invention, nanoparticles are prepared by a process comprising the steps of (a) introducing a selective COX-2 inhibitory drug, a liquid medium, grinding media and a surface modifying agent into a grinding vessel; and (b) wet grinding to reduce the weight average particle size of the drug to less than about 1000 nm, substantially as disclosed in above-cited U.S. Pat. No. 5,565,188 (block copolymer as surface modifying agent containing one or more polyoxyethylene blocks and one or more polyoxy(higher alkylene) blocks wherein at least some of the blocks are linked together by an oxymethylene linking group) and U.S. Pat. No. 5,587,143 (block copolymer of ethylene oxide and butylene oxide as surface modifying agent).

In another related embodiment of the invention, a composition is provided comprising selective COX-2 inhibitory drug nanoparticles having a block copolymer linked to at least one anionic group as a surface modifying agent adsorbed on the surface thereof. The composition is prepared by a process comprising the steps of (a) preparing the drug in particulate form, preferably at a particle size less than about 100 μm; (b) adding the drug to a liquid medium in which it is essentially insoluble to form a premix; and (c) subjecting the premix to mechanical means to reduce the average particle size in the premix to less than about 1000 nm, substantially as disclosed in above-cited U.S. Pat. No. 5,569,448. Preferably, the surface modifying agent is present in the premix.

In another related embodiment of the invention, nanoparticles are prepared by a process comprising the steps of (a) adding a selective COX-2 inhibitory drug and a surface modifying agent (e.g., a steric stabilizer such as gelatin, casein, lecithin, gum acacia, cholesterol, tragacanth, sorbitan esters, polyethylene glycol, polyoxyethylene alkyl esters, polyoxyethylene stearates, etc.) to a liquid in which the drug is insoluble to form a premix, and (b) subjecting the premix to mechanical means (e.g., in a dispersion mill) to reduce average particle size to less than about 400 nm, substantially as disclosed in above-cited U.S. Pat. No. 5,573,783.

In another related embodiment of the invention, nanoparticles are prepared by a process comprising the steps of (a) dispersing a selective COX-2 inhibitory drug and a surface active agent (e.g., poloxamers having a molecular weight of about 1,000 to about 15,000 daltons, polyvinyl alcohol, polyvinylpyrrolidone, hydroxypropylmethylcellulose, and polyoxyethylene sorbitan monooleate) in a liquid dispersion medium in which the drug is poorly soluble, then applying mechanical means (e.g., in a dispersion mill) to reduce drug particle size to less than about 400 nm, substantially as disclosed in above-cited U.S. Pat. No. 5,585,108.

In another related embodiment of the invention, nanoparticles are prepared by a process comprising the steps of (a) adding a selective COX-2 inhibitory drug and hydroxypropylcellulose as a surface modifying agent to a liquid medium in which the drug is essentially insoluble to form a premix, and employing mechanical means (e.g., in a dispersion mill) to reduce drug particle size to less than about 1000 nm, preferably less than about 400 nm, substantially as disclosed in above-cited U.S. Patent No. 5,591,456.

In another related embodiment of the invention, nanoparticles are prepared by a process as described herein that employs a surface modifying agent, the surface modifying agent being selected such that the resulting composition has a hydrophile-lipophile balance (HLB) of about 4 to about 9, substantially as disclosed in above-cited International Patent Publication No. WO 00/30615.

In another particular embodiment of the invention, nanoparticles are prepared by a process comprising the steps of (a) mixing a selective COX-2 inhibitory drug with a support material, preferably a crosslinked, water-swellable polymer; (b) grinding the resulting mixture in a grinding chamber which is saturated with a solvent vapor (e.g., water, ethanol, isopropanol, chloroform, methanol, etc.); (c) drying the ground mixture under vacuum; and (d) sieving the dried ground mixture to eliminate any aggregates formed, substantially as disclosed in above-cited U.S. Pat. No. 5,354,560.

In another particular embodiment of the invention, nanoparticles are prepared by a process comprising the steps of (a) forming a paste comprising (i) nanoparticles of a selective COX-2 inhibitory drug, (ii) at least one thickening or binding agent (e.g., selected from polypeptides, high molecular weight polymers, colloids, etc.) and/or extender, (iii) one or more stabilizing agents to prevent settling and/or rising to the surface of the nanoparticles, and (iv) a suitable amount of water to adjust viscosity; and (b) lyophilizing the paste, substantially as disclosed in above-cited U.S. Pat. No. 5,384,124.

In another particular embodiment of the invention, nanoparticles are prepared by a process comprising the steps of (a) preparing a selective COX-2 inhibitory drug in particulate form, preferably at a particle size smaller than about 100 μm; (b) adding the prepared drug to a liquid medium (preferably comprising a surface modifying agent such as a hygroscopic sugar) in which the drug is essentially insoluble to form a premix; and (c) subjecting the premix to mechanical means to reduce the average particle size in the premix to less than about 1000 nm, substantially as disclosed in above-cited U.S. Pat. No. 5,518,738. Preferably, polyvinylpyrrolidone and/or a wetting agent, e.g., sodium lauryl sulfate, are also present in the premix. Compositions prepared by this process preferably have a film adsorbed on the surface of the nanoparticles comprising a polyvinylpyrrolidone, a hygroscopic sugar and sodium lauryl sulfate.

In another particular embodiment of the invention, nanoparticles are prepared by a process comprising the steps of (a) co-solubilizing one or more polymeric constituents including, for example, a biodegradable polymer (e.g., polylactic acid, polyglycolic acid or co-polymers thereof, polyhydroxybutyric acid, polycaprolactone, polyorthoesters, etc.), a polysaccharide jellifying and/or bioadhesive polymer, and/or an amphiphilic polymer (e.g. polyethylene glycol, polyvinylpyrrolidone or polyvinyl alcohol) together with an agent modifying interface properties to form a polymer mixture, optionally in the presence of one or more solvents; (b) dissolving or suspending a selective COX-2 inhibitory drug in the polymer mixture; and (c) forming particles consisting of the polymers, the agent modifying the interface properties and the drug by a technique of emulsion, extrusion, spray drying or spray congealing, substantially as disclosed in above-cited U.S. Pat. No. 5,536,508. Nanoparticles prepared by this process preferably have a weight average particle size of about 0.1 μm to about 150 μm.

In another particular embodiment of the invention, nanoparticles are prepared by a process comprising the steps of (a) preparing a solution of a selective COX-2 inhibitory drug in a water-miscible organic solvent; (b) infusing an aqueous precipitating liquid (e.g., water, solution of mineral salt, or surfactant solution) into the solution to produce a suspension of precipitated, amorphous, solid drug in the form of non-aggregated particles; and (c) separating the particles from the precipitating liquid and washing in an aqueous washing liquid, substantially as disclosed in above-cited U.S. Pat. No. 4,826,689.

In another particular embodiment of the invention, nanoparticles are prepared by a process comprising the steps of (a) dissolving a selective COX-2 inhibitory drug in an aqueous base (e.g., NaOH, KOH, CsOH, etc.) with stirring to form a solution; (b) adding a surface modifier (e.g., various polymers, surfactants, low molecular weight oligomers, etc.) to form a clear solution; and (c) neutralizing the clear solution with stirring and with an appropriate acid solution (e.g., HCl, HNQ₃, HClO₄, H₂SO₄, formic acid, propionic acid, acetic acid, butyric acid, etc.), substantially as disclosed in above-cited U.S. Pat. Nos. 5,560,932 and No. 5,580,579.

In another related embodiment of the invention, nanoparticles are prepared by a process comprising the steps of (a) dissolving a selective COX-2 inhibitory drug in a liquid medium base (e.g., NaOH, KOH, CsOH, trialkylamines, pyridine, etc.) comprising a non-toxic solvent in which the drug is poorly soluble to form a solution; (b) adding an aqueous solution of one or more surface modifying agents (e.g., anionic or nonionic surfactants, polymeric or oligomeric substances); and (c) neutralizing the resulting alkaline solution with an acid (e.g., HCl, HNO₃, HC10₄, H₂SO₄, formic acid, propionic acid, acetic acid, butyric acid, etc.), to form a dispersion, preferably having a Z-average particle diameter of less than about 100 nm as measured by photon correlation spectroscopy, substantially as disclosed in above-cited U.S. Pat. No. 5,662,883.

In another related embodiment of the invention, nanoparticles are prepared by a process comprising the steps of (a) dissolving a selective COX-2 inhibitory drug and a crystal growth modifier (i.e., a compound that is substantially isostructural to the drug) in an aqueous base (e.g., NaOH, KOH, CsOH, trialkylamines, pyridine, etc.) to form a solution; (b) adding an aqueous solution of one or more surface modifying agents (e.g., a mixture of anionic surfactant, nonionic surfactant, polymeric substance and oligomeric substance); and neutralizing the resulting alkaline solution with an acid (e.g., HCl, HNO₃, HCl0₄, H₂SO₄, formic acid, propionic acid, acetic acid, butyric acid, etc.), to form a dispersion, preferably wherein the drug particles have a Z-average particle diameter of less than about 400 nm as measured by photon correlation spectroscopy, substantially as disclosed in above-cited U.S. Pat. No. 5,665,331.

In another particular embodiment of the invention, nanoparticles having a weight average particle size of less than about 400 nm are prepared from a dispersion comprising a first particle distribution of a selective COX-2 inhibitory drug together with a surface modifying agent such as polysulfated tyloxapol by a process comprising the steps of (a) placing the dispersion between a first electrode and a second electrode; and (b) removing a portion of the dispersion at a position between the first electrode and the second electrode, this portion of the dispersion having a second particle size distribution that is smaller than the first particle distribution, substantially as disclosed in above-cited U.S. Pat. No. 5,503,723.

In another particular embodiment of the invention, nanoparticles having a weight average particle size of up to about 300 nm are prepared by a process comprising the steps of (a) dissolving a selective COX-2 inhibitory drug in a solvent to form a solution; and (b) spraying the solution into a liquefied gas or supercritical fluid in presence of a surface modifying agent dispersed or dissolved in an aqueous phase, substantially as disclosed in above-cited International Patent Publication No. WO 97/14407.

In another related embodiment of the invention, nanoparticles having a weight average particle size of up to about 300 nm are prepared by a process comprising the steps of (a) dissolving a selective COX-2 inhibitory drug in a liquefied gas or supercritical fluid to form a solution; (b) preparing an aqueous phase containing a surface modifying agent; and (c) spraying the solution into the aqueous phase, substantially as disclosed in the same above-cited International Patent Publication No. Wo 97/14407.

Patent and other literature relating to drug nanoparticle compositions teaches that, in general, the smaller the drug particle size, the greater is the advantage in speed of onset of therapeutic effect, or other pharmacodynamic benefit, obtained upon oral administration. For example, at least the following patents propose reduction of particle size to about 400 nm or smaller.

Above-cited U.S. Pat. No. 5,145,684.

Above-cited U.S. Pat. No. 5,298,262.

Above-cited U.S. Pat. No. 5,302,401.

Above-cited U.S. Pat. No. 5,336,507.

Above-cited U.S. Pat. No. 5,340,564.

Above-cited U.S. Pat. No. 5,346,702.

Above-cited U.S. Pat. No. 5,352,459.

Above-cited U.S. Pat. No. 5,429,824.

Above-cited U.S. Pat. No. 5,503,723.

Above-cited U.S. Pat. No. 5,510,118.

Above-cited U.S. Pat. No. 5,534,270.

Above-cited U.S. Pat. No. 5,552,160.

Above-cited U.S. Pat. No. 5,573,783.

Above-cited U.S. Pat. No. 5,585,108.

Above-cited U.S. Pat. No. 5,591,456.

Above-cited U.S. Pat. No. 5,662,883.

Above-cited U.S. Pat. No. 5,665,331.

In general, however, the smaller the drug particle size, the more grinding or milling time, energy and labor is required to produce the particles and consequently, the more costly and less efficient is the process. Thus, smaller nano-sized drug particles are generally significantly more expensive and labor-intensive to produce in quantity than larger nano-sized drug particles.

Surprisingly, we discovered that a selective COX-2 inhibitory drug composition having a weight average particle size of about 450 nm to about 1000 nm (referred to herein as a “sub-micron” formulation and particle size) exhibits onset time and bioavailability substantially equal to that of a comparative composition having a weight average particle size of about 200 to about 400 nm, as measured in vitro and in vivo. The sub-micron formulation requires less milling time and energy than the formulation comprising smaller nanoparticles with a weight average particle size in the 200-400 nm range.

It is further contemplated that certain advantages in addition to cost saving are obtainable with sub-micron as opposed to smaller particle sizes. For example, in situations where ultra-fine particles tend to agglomerate or fail to disperse in the body fluid, the slightly larger sub-micron particles can exhibit enhanced dispersion.

Accordingly, in a particularly preferred embodiment of the present invention, there is provided a pharmaceutical composition comprising a selective COX-2 inhibitory drug of low water solubility in a therapeutically effective amount, wherein the drug is present in solid particles having a D₂₅ particle size of about 450 nm to about 1000 nm, and more preferably about 500 nm to about 900 nm, the composition providing at least a substantially similar Cₘₐₓ and/or at most a substantially similar Tₘₐₓ by comparison with an otherwise similar composition having a D₂₅ particle size of less than 400 nm, and/or providing a substantially greater Cₘₐₓ and/or a substantially shorter Tₘₐₓ by comparison with an otherwise similar composition having a D₂₅ particle size larger than 1000 nm.

There is also provided a pharmaceutical composition comprising a selective COX-2 inhibitory drug of low water solubility in a therapeutically effective amount, wherein the drug is present in solid particles, about 25% to 100% by weight of which have a particle size of about 450 nm to about 1000 nm, more preferably about 500 nm to about 900 nm.

There is also provided a pharmaceutical composition comprising a selective COX-2 inhibitory drug of low water solubility in a therapeutically effective amount, wherein the drug is present in solid particles having a weight average particle size of about 450 nm to about 1000 nm, and more preferably about 500 nm to about 900 nm, the composition providing at least a substantially similar Cₘₐₓ and/or at most a substantially similar Tₘₐₓ by comparison with an otherwise similar composition having a weight average particle size of less than 400 nm, and/or providing a substantially greater Cₘₐₓ and/or a substantially shorter Tₘₐₓ by comparison with an otherwise similar composition having a weight average particle size larger than 1000 nm. For purposes of this description, “weight average particle size” can be considered synonymous with D₅₀ particle size.

One of ordinary skill in the art will readily adapt the processes therein described to the preparation of a poorly water soluble selective cyclooxygenase-2 inhibitory drug, for example celecoxib, deracoxib, valdecoxib, rofecoxib, etoricoxib, 2-(3,5-difluorophenyl)-3-[4-(methylsulfonyl)phenyl]-2-cyclopenten-1-one and 2-(3,4-difluorophenyl)-4-(3-hydroxy-3-methyl-1-butoxy)-5-[4-(methylsulfonyl)phenyl]-3-(2H)-pyridazinone in form of nanoparticles.

In a particular illustrative embodiment of the present invention, the ophthalmic composition comprises an aqueous suspension of a selective COX-2 inhibitory drug of low water solubility, wherein preferably the drug is present predominantly or substantially entirely in form of nanoparticles Without being bound by theory, it is believed that release of the drug from nanoparticles is significantly faster than from a typical “micronized” composition having a D₉₀ particle size of, for example, about 10 μm or greater.

An aqueous suspension composition of the invention can comprise a first portion of the drug in form of nanoparticles, to promote relatively rapid release, and a second portion of the drug having a D₉₀ particle size of about 10 μm or greater, that can provide a depot or reservoir of the drug in the treated eye for release over a period of time, for example about 2 to about 24 hours, more typically about 2 to about 12 hours, to promote sustained therapeutic effect and permit a reduced frequency of administration.

An aqueous suspension can contain one or more polymers as suspending agents. Useful polymers include water-soluble polymers such as cellulosic polymers, e.g., hydroxypropyl methylcellulose, and water-insoluble polymers such as cross-linked carboxyl-containing polymers.

In a particular embodiment the composition is an in situ gellable aqueous solution, suspension or solution/suspension having excipients substantially as disclosed in above-cited U.S. Pat. No. 5,192,535, comprising about 0.1% to about 6.5%, preferably about 0.5% to about 4.5%, by weight, based on the total weight of the composition, of one or more cross-linked carboxyl-containing polymers. Such an aqueous suspension is preferably sterile and has an osmolality of about 10 to about 400 mOsM, preferably about 100 to about 250 mOsM, a pH of about 3 to about 6.5, preferably about 4 to about 6, and an initial viscosity, when administered to the eye, of about 1000 to about 30,000 cPs, as measured at 25° C. using a Brookfield Digital LVT viscometer with #25 spindle and 13R small sample adapter at 12 rpm. More typically the initial viscosity is about 5000 to about 20,000 cPs. The polymer component has an average particle size not greater than about 50 μm, preferably not greater than about 30 μm, more preferably not greater than about 20 μm, and most preferably about 1 μm to about 5 μm, in equivalent spherical diameter, and is lightly cross-linked to a degree such that, upon contact with tear fluid in the eye, which has a typical pH of about 7.2 to about 7.4, the viscosity of the suspension rapidly increases, to form a gel. This formation of a gel enables the composition to remain in the eye for a prolonged period without loss by lacrimal drainage.

Preferred carboxyl-containing polymers for use in this embodiment are prepared from one or more carboxyl-containing monoethylenically unsaturated monomers such as acrylic, methacrylic, ethacrylic, crotonic, angelic, tiglic, α-butylcrotonic, α-phenylacrylic, α-benzylacrylic, α-cyclohexylacrylic, cinnamic, coumaric and umbellic acids, most preferably acrylic acid. The polymers are cross-linked by using less than about 5%, preferably about 0.1% to about 5%, more preferably about 0.2% to about 1%, by weight of one or more polyfunctional cross-linking agents such as non-polyalkenyl polyether difunctional cross-linking monomers, e.g., divinyl glycol. Other suitable cross-linking agents illustratively include 2,3-dihydroxyhexa-1,5-diene, 2,5-dimethylhexa-1,5-diene, divinylbenzene, N,N-diallylacrylamide and N,N-diallylmethacrylamide. Divinyl glycol is preferred. Polyacrylic acid cross-linked with divinyl glycol is called polycarbophil. A polymer system containing polycarbophil is commercially available under the trademark DuraSite® of InSite Vision Inc., Alameda, Calif., as a sustained-release topical ophthalmic delivery system.

A composition of this embodiment can be prepared by a procedure substantially as disclosed in above-cited U.S. Pat. No. 5,192,535. One of skill in the art will readily modify such procedure as appropriate for incorporation of a selective COX-2 inhibitory drug in accordance with the present invention.

In another particular embodiment the composition is an in situ gellable aqueous solution, suspension or solution/suspension having excipients substantially as disclosed in above-cited U.S. Pat. No. 4,861,760, comprising about 0.1% to about 2% by weight of a polysaccharide that gels when it contacts an aqueous medium having the ionic strength of tear fluid. A preferred polysaccharide is gellan gum. A composition of this embodiment can be prepared by a procedure substantially as disclosed in above-cited U.S. Pat. No. 4,861,760. One of skill in the art will readily modify such procedure as appropriate for incorporation of a selective COX-2 inhibitory drug in accordance with the present invention.

In another particular embodiment the composition is an in situ gellable aqueous solution, suspension or solution/suspension having excipients substantially as disclosed in above-cited U.S. Pat. No. 5,587,175, comprising about 0.2% to about 3%, preferably about 0.5% to about 1%, by weight of a gelling polysaccharide, preferably selected from gellan gum, alginate gum and chitosan, and about 1% to about 50% of a water-soluble film-forming polymer, preferably selected from alkylcelluloses (e.g., methylcellulose, ethylcellulose), hydroxyalkylcelluloses (e.g., hydroxyethylcellulose, hydroxypropyl methylcellulose), hyaluronic acid and salts thereof, chondroitin sulfate and salts thereof, polymers of acrylamide, acrylic acid and polycyanoacrylates, polymers of methyl methacrylate and 2-hydroxyethyl methacrylate, polydextrose, cyclodextrins, polydextrin, maltodextrin, dextran, polydextrose, gelatin, collagen, natural gums (e.g., xanthan, locust bean, acacia, tragacanth and carrageenan gums and agar), polygalacturonic acid derivatives (e.g., pectin), polyvinyl alcohol, polyvinylpyrrolidone and polyethylene glycol. The composition can optionally contain a gel-promoting counterion such as calcium in latent form, for example encapsulated in gelatin. A composition of this embodiment can be prepared by a procedure substantially as disclosed in above-cited U.S. Pat. No. 5,587,175. One of skill in the art will readily modify such procedure as appropriate for incorporation of a selective COX-2 inhibitory drug in accordance with the present invention.

In another particular embodiment the composition is an in situ gellable aqueous solution, suspension or solution/suspension having excipients substantially as disclosed in European Patent No. 0 424 043, comprising about 0.1% to about 5% of a carrageenan gum. Carrageenans are sulfated polysaccharides; in this embodiment a carrageenan having no more than 2 sulfate groups per repeating disaccharide unit is preferred, including kappa-carrageenan, having 18-25% ester sulfate by weight, iota-carrageenan, having 25-34% ester sulfate by weight, and mixtures thereof. A composition of this embodiment can be prepared by a procedure substantially as disclosed in above-cited European Patent No. 0 424 043. One of skill in the art will readily modify such procedure as appropriate for incorporation of a selective COX-2 inhibitory drug in accordance with the present invention.

An exemplary formulation of the invention is an ophthalmic suspension of nanoparticles of valdecoxib comprising from about 0.01% to about 50% of valdecoxib, more preferably from about 0.1% to about 20% of valdecoxib, for example from about 0.1% to about 5% of valdecoxib; from about 0.05% to about 10% of carrageenan, preferably from about 0.1% to about 10% of carrageenan, for example from about 0.25% to about 8% of carrageenan and from about 0.5% to about 20% of hydroxypropyl γ-cyclodextrin, preferably from about 1% to about 10% g of hydroxypropyl β-cyclodextrin, for example from about 2% to about 6% of hydroxypropyl β-cyclodextrin (amounts are expressed as % by weight). In another particular embodiment of the invention the composition comprises xanthan gum substantially as disclosed in U.S. Pat. No. 6,174,524.

In another particular embodiment the composition comprises an ophthalmically acceptable mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

In another embodiment of the invention, the selective COX-2 inhibitory drug is solubilized at least in part by an ophthalmically acceptable solubilizing agent. The term “solubilizing agent” herein includes agents that result in formation of a micellar solution or a true solution of the drug. Certain ophthalmically acceptable nonionic surfactants, for example polysorbate 80, can be useful as solubilizing agents, as can ophthalmically acceptable glycols, polyglycols, e.g., polyethylene glycol 400, and glycol ethers.

A class of solubilizing agents having particular utility in solution and solution/suspension compositions of the invention is the cyclodextrins. Suitable cyclodextrins can be selected from α-cyclodextrin, β-cyclodextrin, γ-cyclodextrin, alkylcyclodextrins (e.g., methyl-β-cyclodextrin, dimethyl-β-cyclodextrin, diethyl-β-cyclodextrin), hydroxyalkylcyclodextrins (e.g., hydroxyethyl-β-cyclodextrin, hydroxypropyl-β-cyclodextrin), carboxyalkylcyclodextrins (e.g., carboxymethyl-β-cyclodextrin), sulfoalkylether cyclodextrins (e.g., sulfobutylether-β-cyclodextrin), and the like. Ophthalmic applications of cyclodextrins have been reviewed by Rajewski & Stella (1996), Journal of Pharmaceutical Sciences, 85, 1154, at pages 1155-1159. If desired, complexation of a selective COX-2 inhibitory drug by a cyclodextrin can be increased by addition of a water-soluble polymer such as carboxymethylcellulose, hydroxypropyl methylcellulose or polyvinylpyrrolidone, as described by Loftsson (1998), Pharmazie, 53, 733-740.

One or more ophthalmically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an ophthalmically acceptable range.

The composition of the present invention preferably includes at least one ophthalmically acceptable salt, sugar and/or sugar alcohol in an amount required to bring osmolality of the composition into an ophthalmically acceptable range. Salts suitable for use in the composition include those having sodium, potassium or ammonium cations and chloride, citrate, ascorbate, borate, phosphate, bicarbonate, sulfate, thiosulfate or bisulfite anions; particularly preferred salts include sodium chloride, potassium chloride, sodium thiosulfate, sodium bisulfite and ammonium sulfate, with sodium chloride being especially preferred. Sugars and sugar alcohols suitable for use in the composition include mannitol, dextrose and lactose, glycerol, sorbitol and mannitol; preferred sugars and sugar alcohols include mannitol and dextrose.

The composition optionally includes at least one ophthalmically acceptable acid having at least two dissociable hydrogen as interactive agents to retard release of the drug through inhibition of erosion of the polymer, as disclosed in above-cited International Patent Publication No. WO 95/03784. Acids useful as interactive agents include boric, lactic, orthophosphoric, citric, oxalic, succinic, tartaric and formic glycerophosphoric acids.

The composition optionally includes an ophthalmically acceptable xanthine derivative such as caffeine, theobromine or theophylline, substantially as disclosed in above-cited U.S. Pat. No. 4,559,343, to reduce ocular discomfort associated with administration of the composition.

Optionally, one or more ophthalmically acceptable preservatives are included in the composition to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Optionally, one or more ophthalmically acceptable surfactants, preferably nonionic surfactants are included in the composition to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Optionally, one or more antioxidants are included in the composition to enhance chemical stability where required. Suitable antioxidants include ascorbic acid and sodium metabisulfite.

One or more ophthalmic lubricating agents are optionally included in the composition to promote lacrimation or as a “dry eye” medication. Such agents include polyvinyl alcohol, methylcellulose, hydroxypropyl methylcellulose, polyvinylpyrrolidone, etc.

Compositions of the invention are preferably used in co-therapy with one or more drugs other than selective COX-2 inhibitory drugs. Such drugs other than COX-2 inhibitory drugs can be co-administered topically to the eye together with a composition of the invention. A composition of the invention preferably further comprises, in co-formulation with a first drug that is a selective COX-2 inhibitory drug as described herein, a therapeutically and/or prophylactically effective amount of a second drug that is other than a selective COX-2 inhibitory drug. This second drug cooperates with the first drug in treating and/or preventing a COX-2 mediated ophthalmic condition, or it can be used to treat a related or unrelated condition simultaneously affecting the eye.

Any drug having utility as a topical ophthalmic application can be used in co-therapy, co-administration or co-formulation with a composition of the invention as described immediately above. Such drugs include without limitation demulcents; antibiotics, antivirals and other anti-infectives; steroids, NSAIDs and other anti-inflammatory agents; acetylcholine blocking agents; antiglaucoma agents including beta-adrenergic receptor blocking agents, carbonic anhydrase inhibitors and prostaglandins; antihypertensives; antihistamines; anticataract agents; and topical and regional anesthetics. Illustrative specific drugs include acebutolol, aceclidine, acetylsalicylic acid (aspirin), N⁴ acetylsulfisoxazole, alclofenac, alprenolol, amfenac, amiloride, aminocaproic acid, p-aminoclonidine, aminozolamide, anisindione, apafant, atenolol, bacitracin, benoxaprofen, benoxinate, benzofenac, bepafant, betamethasone, betaxolol, bethanechol, bimatoprost brimonidine, bromfenac, bromhexine, bucloxic acid, bupivacaine, butibufen, carbachol, carprofen, cephalexin, chloramphenicol, chlordiazepoxide, chlorprocaine, chlorpropamide, chlortetracycline, cicloprofen, cinmetacin, ciprofloxacin, clidanac, clindamycin, clonidine, clonixin, clopirac, cocaine, cromolyn, cyclopentolate, cyproheptadine, demecarium, dexamethasone, dibucaine, diclofenac, diflusinal, dipivefrin, dorzolamide, enoxacin, eperezolid, epinephrine, erythromycin, eserine, estradiol, ethacrynic acid, etidocaine, etodolac, fenbufen, fenclofenac, fenclorac, fenoprofen, fentiazac, flufenamic acid, flufenisal, flunoxaprofen, fluorocinolone, fluorometholone, flurbiprofen and esters thereof, fluticasone propionate, furaprofen, furobufen, furofenac, furosemide, gancyclovir, gentamycin, gramicidin, hexylcaine, homatropine, hydrocortisone, ibufenac, ibuprofen and esters thereof, idoxuridine, indomethacin, indoprofen, interferons, isobutylmethylxanthine, isofluorophate, isoproterenol, isoxepac, ketoprofen, ketorolac, labetolol, lactorolac, latanoprost, levobunolol, lidocaine, linezolid, lonazolac, loteprednol, meclofenamate, medrysone, mefenamic acid, mepivacaine, metaproterenol, methanamine, methylprednisolone, metiazinic, metoprolol, metronidazole, minopafant, miroprofen, modipafant, nabumetome, nadolol, namoxyrate, naphazoline, naproxen and esters thereof, neomycin, nepafenac, nitroglycerin, norepinephrine, norfloxacin, nupafant, olfloxacin, olopatadine, oxaprozin, oxepinac, oxyphenbutazone, oxyprenolol, oxytetracycline, penicillins, perfloxacin, phenacetin, phenazopyridine, pheniramine, phenylbutazone, phenylephrine, phenylpropanolamine, phospholine, pilocarpine, pindolol, pirazolac, piroxicam, pirprofen, polymyxin, polymyxin B, prednisolone, prilocaine, probenecid, procaine, proparacaine, protizinic acid, rimexolone, salbutamol, scopolamine, sotalol, sulfacetamide, sulfanilic acid, sulindac, suprofen, tenoxicam, terbutaline, tetracaine, tetracycline, theophyllamine, timolol, tobramycin, tolmetin, travoprost, triamcinolone, trimethoprim, trospectomycin, isopropyl unoprostone, vancomycin, vidarabine, vitamin A, warfarin, zomepirac and pharmaceutically acceptable salts thereof.

In an especially preferred embodiment, a composition of the invention is administered in co-therapy or co-formulation with a prostaglandin capable of reducing the intraocular pressure and/or treating glaucoma. Preferably, the prostaglandin is a derivative of native prostaglandin F_{2α} modified in its omega chain to reduce side effects such a ocular irritation and hyperemia and modified in its alpha chain to improve topical delivery to the cornea. Useful such prostaglandins are described in the patents listed below, each of which is individually incorporated herein by reference.

U.S. Pat. No. 5,422,368.

U.S. Pat. No. 5,001,153.

U.S. Pat. No. 5,515,444.

U.S. Pat. No. 5,510,383.

U.S. Pat. No. 5,665,773.

U.S. Pat. No. 5,889,052.

U.S. Pat. No. 5,352,708.

U.S. Pat. No. 5,607,978.

U.S. Pat. No. 5,658,897.

U.S. Pat. No. 5,688,819.

U.S. Pat. No. 5,834,498.

U.S. Pat. No. 5,972,991.

U.S. Pat. No. 6,037,364.

U.S. Pat. No. 6,124,344.

U.S. Pat. No. 6,160,129.

U.S. Pat. No. 6,204,287.

For example, intraocular pressure reducing prostaglandin derivatives such as latanoprost, travoprost, isopropyl unoprostone and bimatoprost are especially useful. The co-therapy or co-formulation has utility for any of the COX-2 mediated disorders outlined above, in conjunction with glaucoma and/or intraocular hypertension treatment associated with prostaglandins. In addition, co-therapy or co-administration of a selective cyclooxygenase-2 inhibitory drug with a prostaglandin has use in reducing or eliminating side effects that may appear from ocular prostaglandin therapy including, but not limited to, increased iridial pigmentation, disruption of the blood aqueous barrier and cystoid macular edema. Further, the co-therapy or co-administration of a selective cyclooxygenase-2 inhibitory drug and a prostaglandin is of advantage by enabling the extension of a glaucoma or intraocular hypertension prostaglandin treatment to patients suffering from a COX-2 mediated complication during which prostaglandin therapy would otherwise be set out or conducted in conjunction with steroids. Co-therapy or co-administration of a selective cyclooxygenase-2 inhibitory drug and a prostaglandin is also useful in surgical adjunct therapy in connection with eye surgery, e.g., cataract or corneal transplant surgery. This enables or improves glaucoma treatment also in eyes suffering from inflammatory process or trauma, such as cataract surgery, see K Miyake et al Arch. Ophthalmol. 1999, Vol. 117, pages 34-40. The mentioned co-therapy or co-administration is also useful for potentiating the delivery of selective cyclooxygenase-2 inhibitory drugs to exert their activity in the posterior parts of the eye including the region of the optical nerve head. Such delivery enhancing effects of prostagladins have been reported for verapamil in U.S. Pat. No. 5,952,378.

Compositions of the present invention can be prepared by methods known in the art and described in patents and publications cited herein and incorporated herein by reference.

Aqueous suspension compositions of the invention can be packaged in single-dose non-reclosable containers. Such containers can maintain the composition in a sterile condition and thereby eliminate need for preservatives such as mercury-containing preservatives, which can sometimes cause irritation and sensitization of the eye. Alternatively, multiple-dose reclosable containers can be used, in which case it is preferred to include a preservative in the composition.

Formulations of the invention are contemplated to be useful for any drug, of low water solubility, for which ophthalmic administration is desired. Hence, nanoparticle compositions, of any drug of low water solubility, can be formulated substantially as described for selective cyclooxygenase-2 inhibitory drugs herein above. Accordingly, the present invention provides a pharmaceutical composition suitable for topical administration to an eye, the composition comprising nanoparticles of a drug of low water solubility in a concentration effective for treatment and/or prophylaxis of a disorder in the eye, and one or more ophthalmically acceptable excipients that reduce rate of removal of the composition from the eye by lacrimation such that the composition has an effective residence time in the eye of about 2 to about 24 hours.

The present invention, while not limited thereto, will be further illustrated by the following example.

### EXAMPLE

Oral and ophthalmic suspensions of valdecoxib nanoparticles were prepared. The oral nanoparticle suspension formulation contained unlabeled valdecoxib in a concentration of 0.5 mg/g and 2% povidone and 0.15% sodium dodecyl sulfate as vehicle components. The ophthalmic oral nanoparticle suspension formulation contained labeled [¹³C₆] valdecoxib in a concentration of 2.15 mg/g and 1.2% glycerin, 0.8% EDTA.2Na.2H₂O, 4.0% hydroxypropyl β-cyclodextrin (HPBCD), 0.4% carrageenan (Gelcarin™ GP-379NF,FMC Biopolymer, USA), 0.21% carrageenan (SeaSpen™ PF, FMC Biopolymer, USA) and 0.8% povidone. The formulations are shown in Table 1 below. The two carrageenan excipients are both ι-carrageenans with slightly different chemical and physical properties.

TABLE 1

| | Vehicle | Assay |
|---|---|---|
| Formulation | Components | (mg/g ± SD) |
| 0.5 mg/g | 2% Povidone | 0.477 ± 0.009 |
| Valdecoxib | 0.15% Sodium | |
| | Dodecyl Sulfate | |
| 2.5 mg/g [¹³C₆] | 1.2% Glycerin | 2.15 ± 0.03 |
| Valdecoxib | 0.8% | |
| | EDTA.2Na.2H₂O | |
| | 4.0% HPBCD | |
| | 0.4% Gelcarin ™ | |
| | 0.21% Seaspen ™ | |
| | 0.8% Povidone | |

Eighteen male New Zealand white rabbits weighing from 1.8-2.5 kg were provided as test animals and divided into three lots containing six rabbits each. Each of the animals received a single bolus oral dose of unlabeled valdecoxib, in an amount of 0.5 mg/kg of body weight, in the form of the oral unlabeled formulation via gastric intubation. Each of the rabbits also received about 0.1 mg of valdecoxib by applying a single ocular dose of 25 μL of the labeled ophthalmic formulation to each eye of the animal using a 0.5 cc syringe with an attached feeding tube.

A first lot of rabbits was sacrificed 0.5 hours after the administration of the oral and ophthalmic formulations of the valdecoxib, a second lot of rabbits was sacrificed two hours after the drug administration and a third lot of rabbits was sacrificed four hours after the drug administration. Selected eye tissues, aqueous humor, bulbar conjunctiva, cornea, eyelids, vitreous humor and sclera, were excised at the time of sacrifice, weighed directly into tared vials, solubilized and/or diluted as necessary, and analyzed by HPLC-MS. Just prior to sacrifice, 0.5 mL of a blood sample was collected from the ear central artery via a syringe, placed into a K₂-EDTA vacutainer, mixed gently and stored on ice up to one hour prior to centrifuging to separate the plasma. The plasma was stored at −10° C. or colder until analysis.

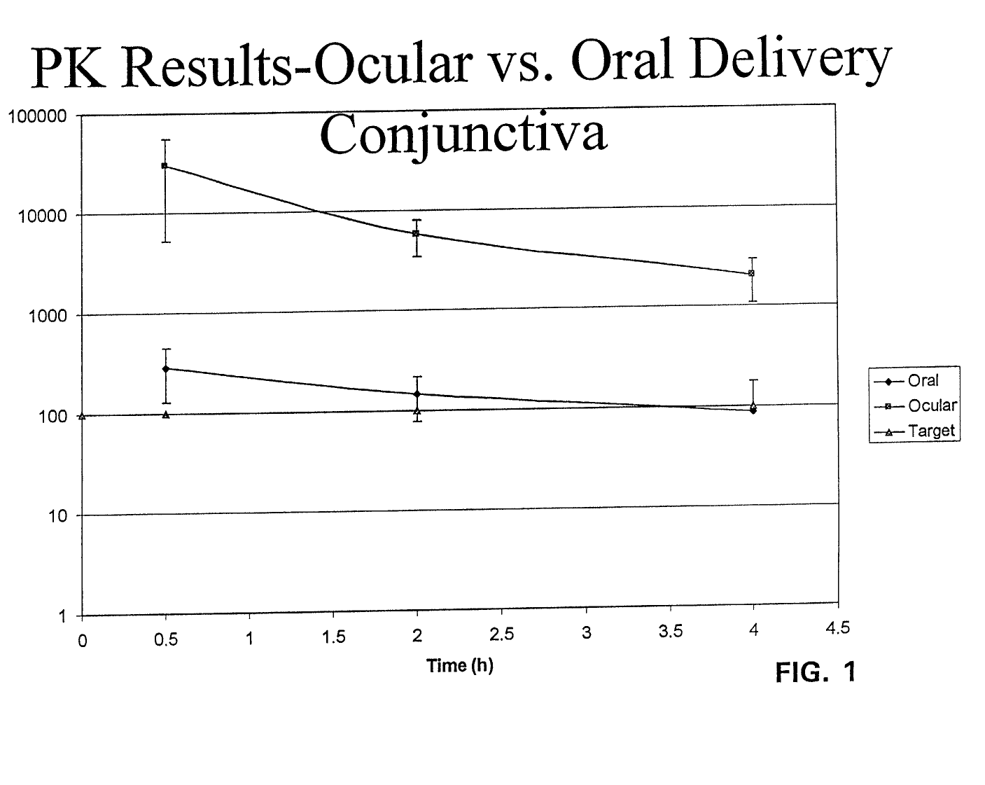

The plasma and eye tissue samples were analyzed by LC-MS and the results are shown in FIGS. 1-3. FIG. 1 is a graph illustrating the pharmacokinetic results of ocular and oral delivery of the valdecoxib formulations plotted as the concentration of the drug in the conjunctiva versus time. As illustrated in FIG. 1, the ocular delivery of the drug achieved a much higher initial concentration of valdecoxib and maintained this higher concentration over time.

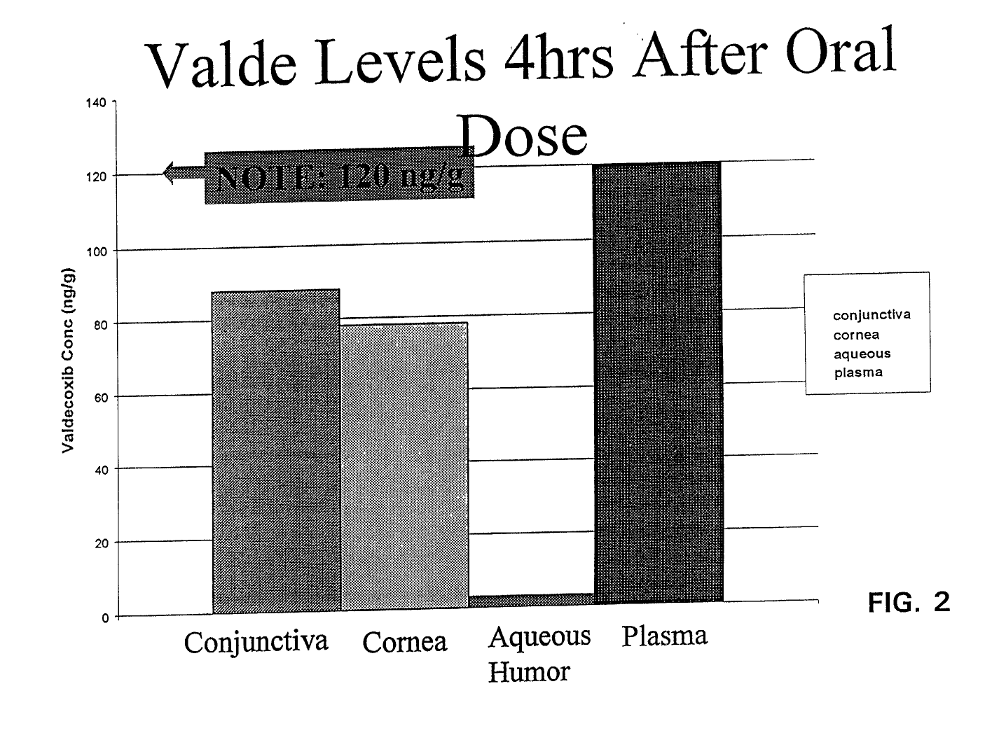

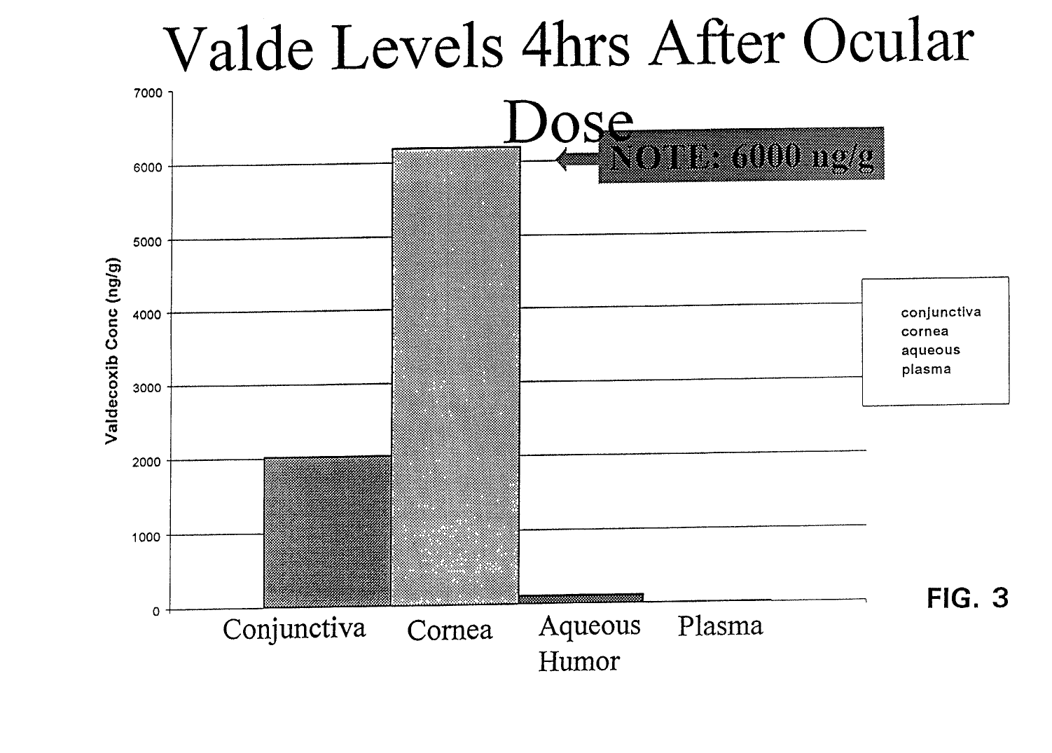

FIG. 2 is a graph illustrating the valdecoxib concentration in the conjunctiva, cornea, aqueous humor and plasma four hours after oral administration and FIG. 3 is a graph illustrating the valdecoxib concentration in the conjunctiva, cornea, aqueous humor and plasma four hours after ocular administration of valdecoxib.

As shown in FIGS. 2 and 3, ocular administration of the valdecoxib is much more effective in delivering the drug to the eye than oral administration and yet avoids the high systemic (plasma) concentration of the drug that accompanies oral delivery.

What is claimed is:

1. A pharmaceutical composition suitable for topical administration to an eye, the composition comprising a selective COX-2 inhibitory drug or a salt or prodrug thereof in a concentration effective for treatment and/or prophylaxis of a COX-2 mediated disorder in the eye, and at least one ophthalmically acceptable excipient ingredient that reduce rate of removal of the composition from the eye by lacrimation such that the composition has an effective residence time in the eye of about 2 to about 24 hours.

2. The composition of claim 1 wherein the selective COX-2 inhibitory drug is of low water solubility.

3. The composition of claim 1 wherein the selective COX-2 inhibitory drug is a compound having the formula: 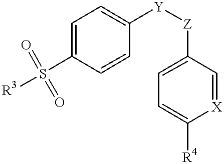
where R3 is a methyl, amino or imide group, R4 is hydrogen or a C1-4 alkyl or alkoxy group, X is N or CR5 where R5 is hydrogen or halogen, and Y and Z are independently carbon or nitrogen atoms defining adjacent atoms of a five- to six-membered ring that is unsubstituted or substituted at one or more positions with oxo, halo, methyl or halomethyl groups; or a prodrug of such a compound.

4. The composition of claim 3 wherein the five- to six-membered ring is a ring selected from the group consisting of: cyclopentenone, furanone, methylpyrazole, isoxazole, and a pyridine ring substituted at no more than one position.

5. The composition of claim 1 wherein the selective COX-2 inhibitory drug is selected from the group consisting of: celecoxib; deracoxib; valdecoxib; rofecoxib; etoricoxib; 2-(3,5-difluorophenyl)-3-[4-(methylsulfonyl)phenyl]-2-cyclopenten-1-one; (S)-6,8-dichloro-2-(trifluoromethyl)-2H-1-benzopyran-3-carboxylic acid; and 2-(3,4-difluorophenyl)-4-(3-hydroxy-3-methyl-1-butoxy)-5-[4-(methylsulfonyl)phenyl]-3-(2H)-pyridazinone.

6. The composition of claim 1 that is in a form selected from the group consisting of: solutions; suspensions and solution/suspensions that are gellable in situ upon placement in the eye; ointments; and solid articles suitable for ocular implant.

7. The composition of claim 1 that is an in situ gellable solution, suspension or solution/suspension having ophthalmically compatible pH and osmolality.

8. The composition of claim 7 that comprises about 0.01% to about 50% weight/volume of the selective COX-2 inhibitory drug.

9. The composition of claim 7 that comprises about 0.1% to about 20% weight/volume of the selective COX-2 inhibitory drug.

10. The composition of claim 7 that is a suspension or solution/suspension wherein the selective COX-2 inhibitory drug is present predominantly as nanoparticles.

11. The composition of claim 10 wherein average particle size of the drug is about 500 to about 900 nm.

12. The composition of claim 7 that (a) comprises about 0.1% to about 6.5% by weight of one or more lightly cross-linked carboxyl-containing polymers, (b) has a pH of about 3 to about 6.5 and an initial viscosity, when administered to the eye, of about 1000 to about 30,000 cPs, and (c) gels on contact with tear fluid having a pH of about 7.2 to about 7.4.

13. The composition of claim 12 wherein the carboxyl-containing polymer is polycarbophil.

14. The composition of claim 7 that comprises about 0.1% to about 2% by weight of a polysaccharide that gels when it contacts an aqueous medium having the ionic strength of tear fluid.

15. The composition of claim 14 wherein the polysaccharide is gellan gum.

16. The composition of claim 7 that comprises about 0.2% to about 3% by weight of a polysaccharide that gels on contact with calcium ions, and about 1% to about 50% of a water-soluble film-forming polymer.

17. The composition of claim 16 wherein the polysaccharide is selected from gellan gum, alginate gum, xanthan gum and chitosan.

18. The composition of claim 7 that comprises an ophthalmically acceptable mucoadhesive polymer.

19. The composition of claim 7 that is a solution or solution/suspension wherein the selective COX-2 inhibitory drug is solubilized at least in part by an ophthalmically acceptable solubilizing agent.

20. The composition of claim 19 wherein the solubilizing agent is a cyclodextrin.

21. The composition of claim 19 wherein the solubilizing agent is polyethylene glycol.

22. The composition of claim 10 comprising from about 0.01% to about 50% by weight of valdecoxib, from about 0.05% to about 10% by weight of carrageenan, and from about 0.5% to about 20% by weight of hydroxypropyl β-cyclodextrin.

23. A method of treating and/or preventing a COX-2 mediated disorder in an eye of a mammalian subject, the method comprising administering in each of one or more topical applications to the eye a therapeutically or prophylactically effective amount of a composition comprising a selective COX-2 inhibitory drug or a salt or prodrug thereof and one or more ophthalmically acceptable excipient ingredients that reduce rate of removal of the composition from the eye by lacrimation such that the composition has an effective residence time in the eye of about 2 to about 24 hours.

24. The method of claim 23 wherein the mammalian subject is a human subject.

25. The method of claim 24 wherein the selective COX-2 inhibitory drug is a compound having the formula: 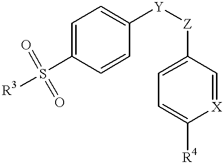
where R3 is a methyl or amino group, R4 is hydrogen or a C1-4 alkyl or alkoxy group, X is N or CR5 where R5 is hydrogen or halogen, and Y and Z are independently carbon or nitrogen atoms defining adjacent atoms of a five- to six-membered ring that is unsubstituted or substituted at one or more positions with oxo, halo, methyl or halomethyl groups; or a prodrug of such a compound.

26. The method of claim 25 wherein the five- to six-membered ring is selected from the group consisting of: cyclopentenone, furanone, methylpyrazole, isoxazole and pyridine rings substituted at no more than one position.

27. The method of claim 24 wherein the selective COX-2 inhibitory drug is selected from the group consisting of: celecoxib, deracoxib, valdecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid and 2-(3,4-difluorophenyl)-4-(3-hydroxy-3-methyl-1-butoxy)-5-[4-(methylsulfonyl)phenyl]-3-(2H)-pyridazinone.

28. The method of claim 24 that further comprises topical administration to the eye, in co-therapy, co-administration and/or co-formulation with the selective COX-2 inhibitory drug, a second drug.

29. The method of claim 28 wherein the second drug is selected from acebutolol, aceclidine, acetylsalicylic acid, N4 acetylsulfisoxazole, alclofenac, alprenolol, amfenac, amiloride, aminocaproic acid, p-aminoclonidine, aminozolamide, anisindione, apafant, atenolol, bacitracin, benoxaprofen, benoxinate, benzofenac, bepafant, betamethasone, betaxolol, bethanechol, bimatoprost, brimonidine, bromfenac, bromhexine, bucloxic acid, bupivacaine, butibufen, carbachol, carprofen, cephalexin, chloramphenicol, chlordiazepoxide, chlorprocaine, chlorpropamide, chlortetracycline, cicloprofen, cinmetacin, ciprofloxacin, clidanac, clindamycin, clonidine, clonixin, clopirac, cocaine, cromolyn, cyclopentolate, cyproheptadine, demecarium, dexamethasone, dibucaine, diclofenac, diflusinal, dipivefrin, dorzolamide, enoxacin, eperezolid, epinephrine, erythromycin, eserine, estradiol, ethacrynic acid, etidocaine, etodolac, fenbufen, fenclofenac, fenclorac, fenoprofen, fentiazac, flufenamic acid, flufenisal, flunoxaprofen, fluorocinolone, fluorometholone, flurbiprofen and esters thereof, fluticasone propionate, furaprofen, furobufen, furofenac, furosemide, gancyclovir, gentamycin, gramicidin, hexylcaine, homatropine, hydrocortisone, ibufenac, ibuprofen and esters thereof, idoxuridine, indomethacin, indoprofen, interferons, isobutylmethylxanthine, isofluorophate, isoproterenol, isoxepac, ketoprofen, ketorolac, labetolol, lactorolac, latanoprost, levobunolol, lidocaine, linezolid, lonazolac, loteprednol, meclofenamate, medrysone, mefenamic acid, mepivacaine, metaproterenol, methanamine, methylprednisolone, metiazinic, metoprolol, metronidazole, minopafant, miroprofen, modipafant, nabumetome, nadolol, namoxyrate, naphazoline, naproxen and esters thereof, neomycin, nepafenac, nitroglycerin, norepinephrine, norfloxacin, nupafant, olfloxacin, olopatadine, oxaprozin, oxepinac, oxyphenbutazone, oxyprenolol, oxytetracycline, penicillins, perfloxacin, phenacetin, phenazopyridine, pheniramine, phenylbutazone, phenylephrine, phenylpropanolamine, phospholine, pilocarpine, pindolol, pirazolac, piroxicam, pirprofen, polymyxin, polymyxin B, prednisolone, prilocaine, probenecid, procaine, proparacaine, protizinic acid, rimexolone, salbutamol, scopolamine, sotalol, sulfacetamide, sulfanilic acid, sulindac, suprofen, tenoxicam, terbutaline, tetracaine, tetracycline, theophyllamine, timolol, tobramycin, tolmetin, travoprost, triamcinolone, trimethoprim, trospectomycin, isopropyl unoprostone, vancomycin, vidarabine, vitamin A, warfarin, zomepirac and pharmaceutically acceptable salts thereof.

30. The method of claim 28 wherein the second drug is a prostaglandin.

31. The method of claim 30 where iridial pigmentation after administration to an eye is reduced by comparison with treatment with the prostaglandin alone.

32. The method of claim 30 where the drugs are administrated as surgical adjunct therapy in connection with eye surgery.

33. The method of claim 30 wherein the prostaglandin is selected from latanoprost, bimatoprost travoprost and isopropyl unoprostone.

34. The method of claim 24 wherein the composition is an in situ gellable solution, suspension or solution/suspension having ophthalmically compatible pH and osmolality.

35. The method of claim 24 wherein the selective COX-2 inhibitory drug is present predominantly as nanoparticles.

36. The method of claim 35 wherein average particle size of the drug is about 500 to 900 nm.

37. A method of manufacturing a medicament for topically treating or preventing a COX-2 mediated disorder in an eye, comprising a step of utilizing a composition comprising a selective COX-2 inhibitory drug or a salt or prodrug thereof and one or more ophthalmically acceptable excipient ingredients that reduce rate of removal of the composition from the eye by lacrimation such that the composition has an effective residence time in the eye of about 2 to about 24 hours in the medicament.

38. The method of claim 23 wherein the drug is administered by electroosmosis, electroporation or by iontophoresis.

39. A pharmaceutical composition suitable for topical administration to an eye, the composition comprising nanoparticles of a drug of low water solubility in a concentration effective for treatment and/or prophylaxis of a disorder in the eye, and one or more ophthalmically acceptable excipients that reduce rate of removal of the composition from the eye by lacrimation such that the composition has an effective residence time in the eye of about 2 to about 24 hours.

40. A method of treating and/or preventing a disorder in an eye of a mammalian subject, the method comprising administering in each of one or more topical applications to the eye a therapeutically or prophylactically effective amount of a composition comprising nanoparticles of a drug of low water solubility and one or more ophthalmically acceptable excipients that reduce rate of removal of the composition from the eye by lacrimation such that the composition has an effective residence time in the eye of about 2 to about 24 hours.
